(19) United States
(12) Patent Application Publication
(10) Pub. No.: US 2011/0023881 A1
(43) Pub. Date: Feb. 3, 2011

(54) Method And System For Generating A Pressure Volume Loop Of A Low Flow Recruitment Maneuver

(75) Inventor: Ron Thiessen, Mission (CA)

Correspondence Address: NELLCOR PURITAN BENNETT LLC, 6135 GUNBARREL AVENUE, BOULDER, CO, 80301, US

(73) Assignee: Nellcor Puritan Bennett LLC, Boulder, CO (US)

(21) Appl. No.: 12/833,706

(22) Filed: Jul. 9, 2010

Related U.S. Application Data
(60) Provisional application No. 61/230,430, filed on Jul. 31, 2009.

(51) Int. Cl. A61M 16/00

(52) U.S. Cl. 128/204.23; 128/204.21

(57) ABSTRACT

This disclosure describes systems and methods for delivering one or more low flow recruitment maneuvers to a patient while on a ventilator. Embodiments described herein provide methods for delivering low flow recruitment maneuvers wherein either or both of the inspiratory and expiratory phases of the recruitment maneuver are maintained by the ventilator at a low flow. Embodiments described herein provide for single-breath recruitment maneuvers and multi-breath recruitment maneuvers at low flow. Embodiments described herein provide for graphical display of a pressure-volume loop (PV loop) for both single-breath and multi-breath recruitment maneuvers. Embodiments described herein also disclose an automated ventilator functionality whereby recruitment maneuvers settings and/or post-recruitment maneuver settings for resuming prescribed ventilation may be set via a graphical user interface. Other embodiments described herein enable a clinician to configure the ventilator to synchronize the end the recruitment maneuver with patient-triggered inspiration for post-recruitment maneuver ventilation.

### RELATED APPLICATIONS

This application claims priority to U.S. Provisional Patent Application Ser. No. 61/230,430, entitled “METHOD AND SYSTEM FOR GENERATING A PRESSURE VOLUME LOOP OF A LOW FLOW RECRUITMENT MANEUVER,” filed on Jul. 31, 2009, the entire disclosure of which is hereby incorporated herein by reference.

### INTRODUCTION

A ventilator is a device that mechanically helps patients breathe by replacing some or all of the muscular effort required to inflate and deflate the lungs. Ventilatory assistance is indicated for certain diseases affecting the musculature required for breathing, such as muscular dystrophies, polio, amyotrophic lateral sclerosis (ALS), and Guillain-Barré syndrome. Mechanical ventilation may also be required during the sedation associated with surgery and as the result of various injuries, such as high spinal cord injuries and head traumas.

Ventilators may provide assistance according to a variety of methods based on the needs of the patient. These methods include volume-cycled and pressure-cycled methods. Specifically, volume-cycled methods may include among others, Pressure-Regulated Volume Control (PRVC), Volume Ventilation (VV), and Volume Controlled Continuous Mandatory Ventilation (VC-CMV) techniques. Pressure-cycled methods may involve, among others, Assist Control (AC), Synchronized Intermittent Mandatory Ventilation (SIMV), Controlled Mechanical Ventilation (CMV), Pressure Support Ventilation (PSV), Continuous Positive Airway Pressure (CPAP), or Positive End Expiratory Pressure (PEEP) techniques.

In addition to providing breathing assistance to a patient, a ventilator may also be configured to expand non-functioning regions of a patient's lung(s). Specifically, the ventilator may expand collapsed alveoli so that the functional surface area of the patient's lung(s) is thereby increased. The process by which the ventilator “recruits” collapsed alveoli is called a “recruitment maneuver.”

Currently, the optimal method of delivery is debatable and, as recruitment maneuvers are only occasionally delivered during ventilation, the complex settings for delivering recruitment maneuvers are manually configured on ventilators by clinicians.

### Method and System for Generating a Pressure Volume Loop of a Low Flow Recruitment Maneuver

This disclosure describes systems and methods for delivering one or more low flow recruitment maneuvers to a patient while on a ventilator. In general, the purpose of a recruitment maneuver is to expand collapsed alveoli in order to increase the functional surface area of a patient's lungs. The present disclosure may be applicable for adult, pediatric, and neonatal ventilatory techniques, as required by the diverse care plans of various patients.

Ventilators may be configured to deliver recruitment maneuvers by a variety of methods. For example, there may be four general types of recruitment maneuvers that may be advantageous for reclaiming collapsed alveoli. A “normal flow” single-breath recruitment maneuver may be described as a single-breath recruitment maneuver delivered at conventional flows (e.g., 30-100 liters per minute, lpm) and may be indicated when recruitment is desired but a lung compliance assessment based on a low flow pressure-volume curve is not desired. A “low flow” single-breath recruitment maneuver may be described as a single breath recruitment maneuver that is delivered at low flow (e.g., less than 10 lpm) and may be indicated when both recruitment and a compliance assessment are desired. A “normal flow” multi-breath recruitment maneuver may be described as a recruitment maneuver delivered over multiple breaths at conventional flows and may be indicated when recruitment, but not a compliance assessment, is desired. Finally, a “low flow” multi-breath recruitment maneuver may be described as a multiple-breath recruitment delivered at low flow where both recruitment and compliance assessment are desired.

Healthy alveoli are coated with a viscous fluid that lubricates and protects the alveoli and promotes gas exchange. Normally, this fluid allows the alveoli to expand and contract without adhering to one another. Healthy alveoli do not fully contract during expiration; but rather, a base volume of air remains in the alveoli at the end of expiration to prevent the interior surfaces of the alveoli from adhering together. Disease or other adverse conditions, however, may cause the viscous fluid to become tacky, gluing alveoli to one another and/or gluing the interior surfaces of the alveoli together. When alveoli completely collapse, e.g., as the result of disease or a surgical procedure, it is very difficult to pry them open again. Thus, it may be necessary to deliver significant pressure, for a significant length of time, to force collapsed alveoli open again during a recruitment maneuver (RM).

Studies have indicated, however, that recruitment maneuvers may injure the delicate brachial and alveolar structures of the lungs when this heightened pressure is delivered or released too quickly. As will be discussed further below, the airflow into and out of the lungs is governed by a pressure gradient between the lungs and the external atmospheric pressure. The greater the pressure gradient, the greater the resultant flow into or out of a patient's lungs. The present disclosure proposes a recruitment maneuver conducted at low flow; as for instance, flow maintained at less than 10 liters per minute.

Embodiments described herein seek to provide methods for delivering low flow recruitment maneuvers wherein either or both of the inspiratory and expiratory phases of the recruitment maneuver are maintained by the ventilator at a low flow. Embodiments described herein provide for a single-breath recruitment maneuver at low flow and also for a multi-breath recruitment maneuver at low flow.

Embodiments described herein provide for graphical display of a pressure-volume loop (PV loop) for both single-breath recruitment maneuvers and multi-breath recruitment maneuvers. Additionally, embodiments provide for comparing a recruitment maneuver PV loop with previous PV loops calculated during the prescribed ventilation of a patient.

Embodiments described herein also allow for an automated ventilator functionality whereby recruitment maneuvers may be pre-set by clinicians. Specifically, the clinician may input particular patient-specific parameters, e.g., positive-end expiratory pressure (PEEP), a peak inspiratory pressure (or, target recruitment maneuver pressure), a duration of hold for the target recruitment maneuver pressure, FiO₂, and a peak targeted flow to be maintained during the recruitment maneuver. In response to the clinician-defined parameters, the ventilator may automatically deliver the recruitment maneuver at a prescribed inspiratory and expiratory low flow, i.e., less than or equal to the peak targeted flow (e.g., 10 liters per minute (lpm)).

Embodiments described herein also disclose a ventilator system wherein the user interface may be configured to call and display a “Recruitment Maneuver Input” screen from a general ventilation input screen. The Recruitment Maneuver Input screen may provide various windows and elements whereby a clinician may input parameters for delivering one or more recruitment maneuvers. The Recruitment Maneuver Input screen may be further configured to allow a clinician to save recruitment maneuver settings on a per patient basis. Prior to delivering each recruitment maneuver, the clinician may be prompted with an option to deliver the recruitment maneuver according to previous settings or an option to deliver the recruitment maneuver according to manually entered settings. The Recruitment Maneuver Input screen may also include an “Initiate Recruitment Maneuver” selection and a “Recruitment Maneuver Interrupt” selection.

Embodiments described herein also disclose a ventilator system wherein the Recruitment Maneuver Input screen allows a clinician to indicate post-recruitment maneuver settings for resuming prescribed ventilation. The clinician may select an option to resume ventilation according to pre-recruitment maneuver settings or, optionally, to reconfigure the ventilator to deliver prescribed ventilation according to alternate post-recruitment maneuver settings.

Embodiments described herein also enable a clinician to differentiate the timing of a recruitment maneuver depending on whether a patient is sedated or paralyzed or whether a patient is able to breathe spontaneously. Wherein the patient is able to breathe spontaneously, the ventilator may be configured to synchronize the end of the recruitment maneuver before such spontaneously breathing patient initiates an inspiratory breath of the prescribed ventilation.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of described technology and are not meant to limit the scope of the invention as claimed in any manner, which scope shall be based on the claims appended hereto.

FIG. 1 is a diagram illustrating a representative ventilator system utilizing an endrotracheal tube for air delivery to a patient's lungs.

FIG. 2 is a block-diagram illustrating an embodiment of a ventilatory system for delivering a single-breath, low flow recruitment maneuver as described herein.

FIG. 3 is an illustration of an embodiment of a graphical user interface for receiving clinician input for a single-breath, low flow recruitment maneuver as described herein.

FIG. 4 is a flow-diagram illustrating an embodiment of a method for delivering a single-breath, low flow recruitment maneuver as described herein.

FIG. 5 is a flow-diagram illustrating an embodiment of a method for delivering an inspiratory phase of a single-breath, low flow recruitment maneuver as described herein.

FIG. 6 is a flow-diagram illustrating an embodiment of a method for delivering a single-breath, low flow recruitment maneuver incorporating a hold time as described herein.

FIG. 7 is a flow-diagram illustrating an embodiment of a method for delivering an expiratory phase of a single-breath, low flow recruitment maneuver as described herein.

FIG. 8 is a block-diagram illustrating an embodiment of a ventilatory system for delivering a multi-breath, low flow recruitment maneuver as described herein.

FIG. 9 is an illustration of an embodiment of a graphical user interface for receiving clinician input for a multi-breath, low flow recruitment maneuver as described herein.

FIGS. 10a and 10h are flow-diagrams illustrating an embodiment of a method for delivering breaths in an ascending phase of a multi-breath, low flow recruitment maneuver as described herein.

FIGS. 11a and 11b are flow-diagrams illustrating an embodiment of a method for delivering breaths in a descending phase of a multi-breath, low flow recruitment maneuver as described herein.

FIG. 12 is a flow-diagram illustrating an embodiment of a method for synchronizing the end of a recruitment maneuver with the initiation of post-recruitment maneuver prescribed ventilation.

FIG. 13 is a flow-diagram illustrating an embodiment of a method for generating a pressure-volume loop during a single-breath, low flow recruitment maneuver as described herein.

FIG. 14 is a flow-diagram illustrating an embodiment of a method for generating a pressure-volume loop during a multi-breath, low flow recruitment maneuver as described herein.

### DETAILED DESCRIPTION

Although the techniques introduced above and discussed in detail below may be implemented for a variety of medical devices, the present disclosure will discuss the implementation of these techniques for use in a mechanical ventilator system. The reader will understand that the technology described in the context of a ventilator system could be adapted for use with other systems in which gas volume, pressure, and flow should be carefully regulated.

This disclosure describes systems and methods for delivering one or more low flow recruitment maneuvers to a patient while on a ventilator. As described above, recruitment maneuvers are delivered to a patient to expand collapsed alveoli in order to increase the functional surface area of a patient's lungs. Recruitment maneuvers may be particularly useful for a patient who suffers from collapsed alveoli due to pneumonia or Acute Respiratory Distress Syndrome (ARDS), i.e., any disease or condition in which portions of the lung are inactive due to collapsed alveoli.

As mentioned previously, when the alveoli collapse the interior surfaces of the alveoli may adhere together. Thus, significant pressure, for a significant length of time, may be generally necessary to force collapsed alveoli open again during a recruitment maneuver. This “significant pressure” may be described herein as the “target recruitment maneuver pressure.” The “significant length of time” may be described herein as the recruitment maneuver “hold time.”

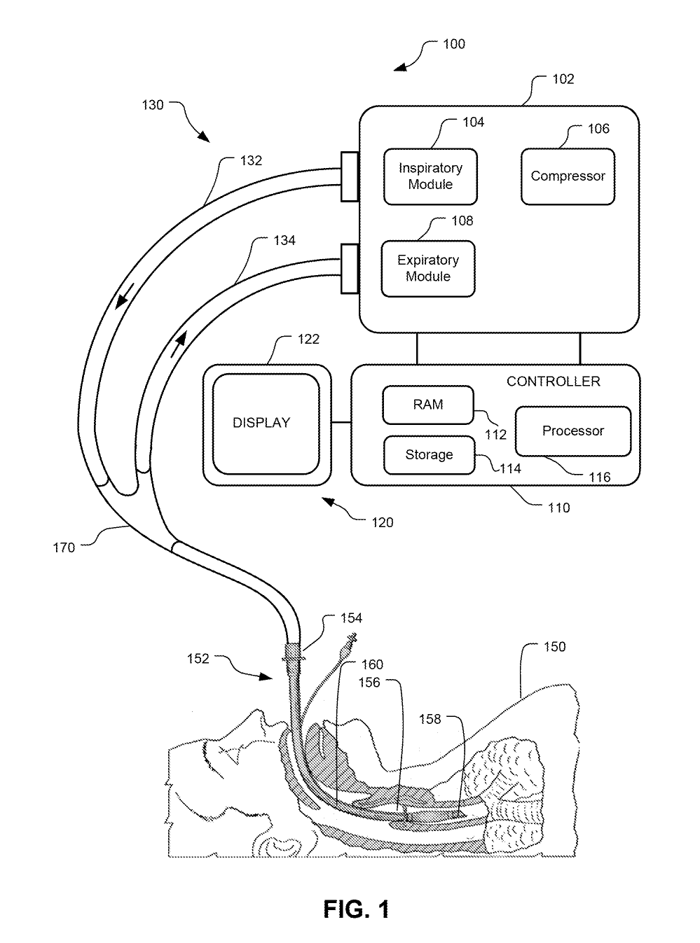

FIG. 1 illustrates an embodiment of a ventilator 100 connected to a human patient 150. Ventilator 100 includes a pneumatic system 102 (also referred to as a pressure generating system 102) for circulating breathing gases to and from patient 150 via the ventilation tubing system 130, which couples the patient to the pneumatic system via an invasive patient interface 152.

Ventilation may be achieved by invasive or non-invasive means. Invasive ventilation, such as invasive patient interface 152, utilizes a breathing tube, particularly an endrotracheal tube (ET tube) or a tracheotomy tube (trach tube), inserted into the patient's trachea in order to deliver air to the lungs. Non-invasive ventilation may utilize a mask or other device placed over the patient's nose and mouth. For the purposes of this disclosure, an invasive patient interface 152 is shown and described, although the reader will understand that the technology described herein is equally applicable to any invasive or non-invasive patient interface.

Airflow is provided via ventilation tubing circuit 130 and invasive patient interface 152. Ventilation tubing circuit 130 may be a dual-limb (shown) or a single-limb circuit for carrying gas to and from the patient 150. In a dual-limb embodiment as shown, a “wye fitting” 170 may be provided to couple the patient interface 154 to an inspiratory limb 132 and an expiratory limb 134 of the ventilation tubing circuit 130.

Pneumatic system 102 may be configured in a variety of ways. In the present example, system 102 includes an expiratory module 108 coupled with the expiratory limb 134 and an inspiratory module 104 coupled with the inspiratory limb 132. Compressor 106 or another source(s) of pressurized gases (e.g., air, oxygen, and/or helium) is coupled with inspiratory module 104 to provide a gas source for ventilatory support via inspiratory limb 132.

The pneumatic system may include a variety of other components, including sources for pressurized air and/or oxygen, mixing modules, valves, sensors, tubing, accumulators, filters, etc. Controller 110 is operatively coupled with pneumatic system 102, signal measurement and acquisition systems, and an operator interface 120 may be provided to enable an operator to interact with the ventilator 100 (e.g., change ventilator settings, select operational modes, view monitored parameters, etc.). Controller 110 may include memory 112, one or more processors 116, storage 114, and/or other components of the type commonly found in command and control computing devices.

The memory 112 is computer-readable storage media that stores software that is executed by the processor 116 and which controls the operation of the ventilator 100. In an embodiment, the memory 112 includes one or more solid-state storage devices such as flash memory chips. In an alternative embodiment, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. Computer-readable storage media includes volatile and non-volatile, removable and non-removable media implemented in any method or technology for storage of information such as computer-readable instructions, data structures, program modules or other data. Computer-readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

As described in more detail below, controller 110 issues commands to pneumatic system 102 in order to control the breathing assistance provided to the patient by the ventilator. The specific commands may be based on inputs received from patient 150, pneumatic system 102 and sensors, operator interface 120 and/or other components of the ventilator. In the depicted example, operator interface includes a display 122 that is touch-sensitive, enabling the display to serve both as an input and output device.

As will be described further herein, the controller 110 may be configured to deliver recruitment maneuvers on a set schedule according to automated settings based on patient-specific parameters or manual settings entered by a clinician and/or pre-configured and saved by the clinician.

### Embodiments of Single-Breath Recruitment Maneuvers

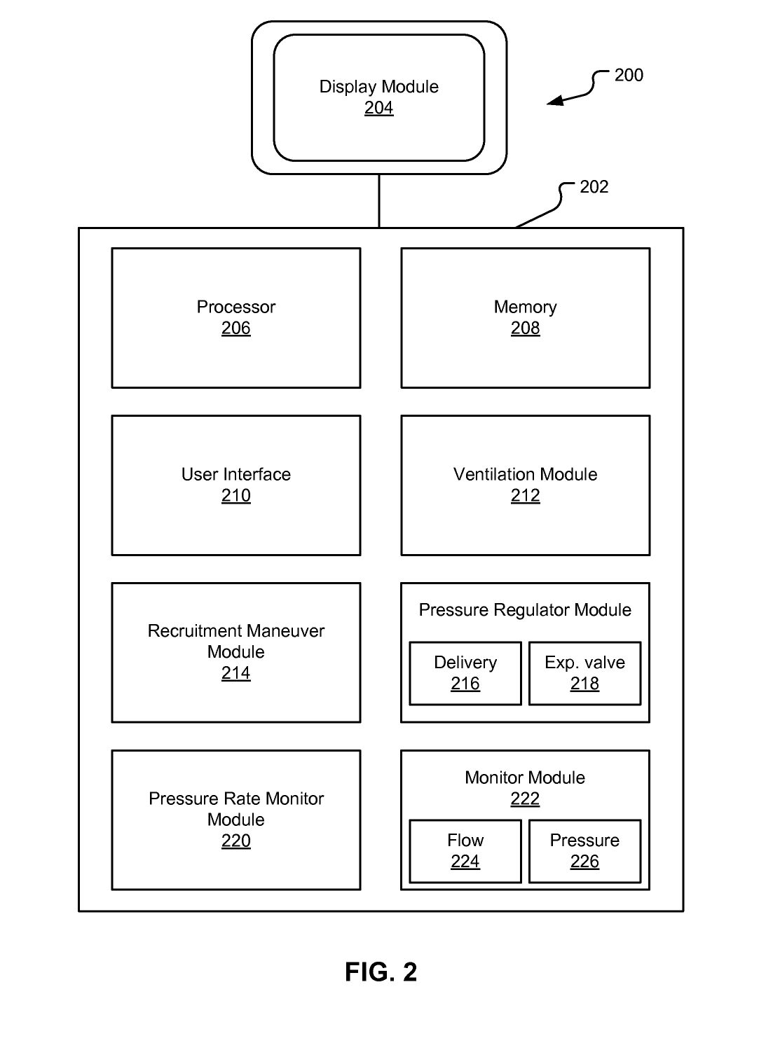

FIG. 2 is a block-diagram illustrating an embodiment of a ventilatory system 200 for delivering a single-breath, low flow recruitment maneuver as described herein.

The ventilator 202 includes a display module 204, memory 208, one or more processors 206, user interface 210, and various ventilation and recruitment maneuver modules 212-226. Memory 208 is defined as described above for memory 112. Similarly, the one or more processors 206 are defined as described above for the one or more processors 116. Processors 206 may further be configured with a clock whereby elapsed time may be monitored by the system 200.

The display module 204 displays various input screens to a clinician, including but not limited to a “Recruitment Maneuver Input Screen,” as will be described further herein, for receiving clinician input. The display module 204 is configured to communicate with user interface 210. Specifically, display module 204 and user interface 210 may receive recruitment maneuver setting information from a clinician, as described further herein. The user interface 210 may provide various windows and elements to the clinician for input and interface command options. Additionally, user interface 210 may provide useful information to the clinician through display module 204. This useful information may be in the form of various clinical data regarding the patient, displaying for instance, the patient's FiO₂ level, peak inspiratory pressure (PIP), and positive end expiratory pressure (PEEP), etc. Alternately, useful information may be derived by the ventilator 202, based on data gathered from the various ventilation and recruitment maneuver modules 212-226, and the useful information may be displayed in the form of graphs, wave representations, or other suitable forms of display to the clinician. Examples of such graphic representations may include, but are not limited to, pressure and volume curves, flow curves, and pressure-volume loops, etc. Display module 204 may further be an interactive display, whereby the clinician may both receive and communicate information to the ventilator 202, as by a touch-activated display screen. Alternately, user interface 210 may provide other suitable means of communication with the ventilator 202, for instance by a keyboard or other suitable interactive device.

Ventilation module 212 oversees prescribed ventilation as delivered to a patient. Specifically, prescribed ventilation refers to the ventilatory settings for the patient during routine ventilation, i.e., when the patient is not receiving a recruitment maneuver. Prescribed ventilation may be delivered according to uniform settings, both before and after a recruitment maneuver. Alternately, a clinician may wish to alter ventilatory treatment after a recruitment maneuver, wherein one or more pre-recruitment maneuver settings may be different from one or more post-recruitment maneuver settings. Embodiments of the present system may also allow for recruitment maneuvers to be synchronized with post-recruitment maneuver prescribed ventilation, for example, by timing an end of the expiratory phase of a single-breath recruitment maneuver with a beginning of a subsequent inspiratory phase of the prescribed ventilation. Alternately, each breath of a multi-breath recruitment maneuver may be synchronized to coincide with triggers initiated by the patient, or the final breath of a multi-breath recruitment maneuver may be synchronized with the beginning of a subsequent inspiratory phase of prescribed ventilation. Synchronization may be indicated, for instance, where the patient is a spontaneously breathing patient, as described further herein.

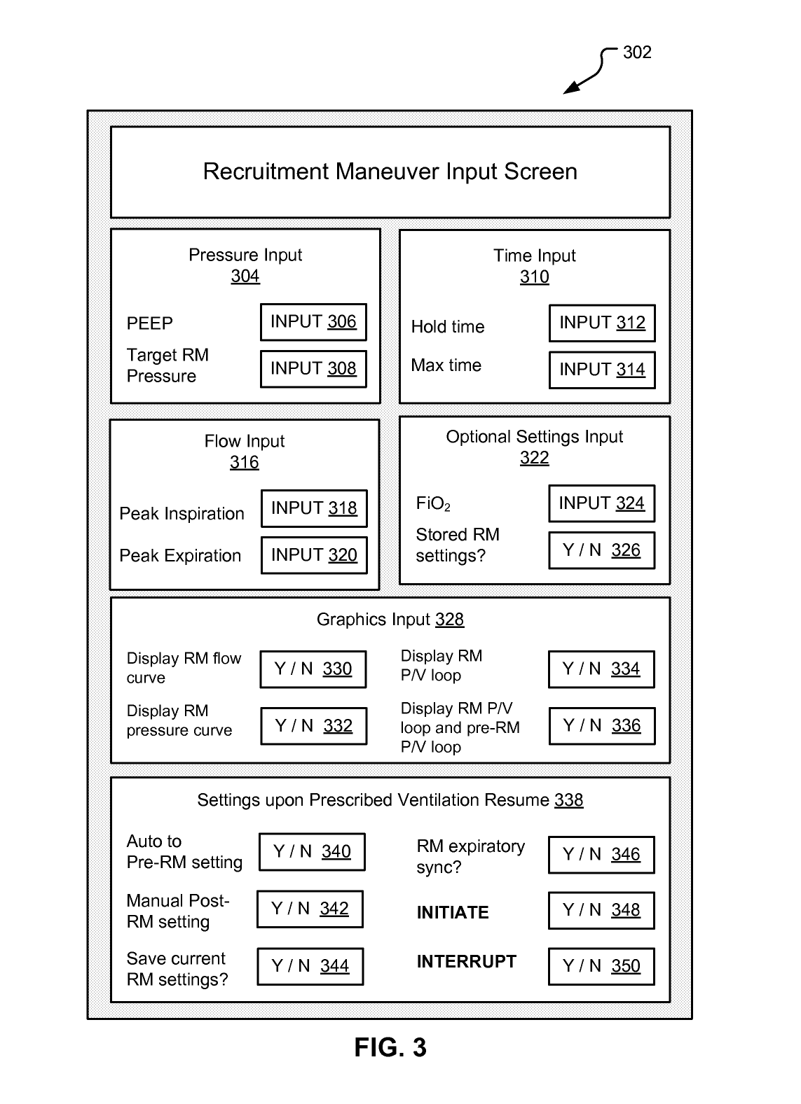

In ventilator 202, a recruitment maneuver module 214 may oversee single-breath recruitment maneuvers as delivered to a patient. Specifically, recruitment maneuver module 214 may deliver a single-breath recruitment maneuver according to settings input by a clinician or, alternately, according to default settings pre-configured according to a standard protocol, for instance a hospital-specific or physician-specific protocol. As will be described further herein with reference to FIG. 3, the clinician may configure recruitment maneuver settings by calling on a single-breath “Recruitment Maneuver Input Screen” from a main ventilation input display. Input for a single-breath recruitment maneuver may include, but is not limited to, input regarding a target recruitment maneuver pressure. Input may further include settings for a baseline pressure (described further below), a hold time for the recruitment maneuver target pressure, a peak targeted flow, and settings for delivering post-recruitment maneuver prescribed ventilation. Clinician input may be received at any suitable time during or before the prescribed ventilation and before initiating a recruitment maneuver. For instance, clinician input may be received immediately before initiating the recruitment maneuver or during initial ventilator set-up.

Specifically, a recruitment maneuver module 214 may be in communication with the various other modules described below to oversee the complete delivery of the recruitment maneuver. For example, the recruitment maneuver module 214 may initiate the single-breath low flow recruitment maneuver; transition from one phase of the recruitment maneuver to a next phase (e.g., transitioning from the inspiratory phase to the hold phase, etc.); terminate the recruitment maneuver; and transition back into the prescribed ventilation. The recruitment maneuver module 214 may also orchestrate and supervise any number of other functions in order to provide a single-breath recruitment maneuver to a patient.

The recruitment maneuver module 214 may be further configured to ignore existing alarm settings during delivery of a recruitment maneuver. For example, volume, pressure, flow, and apnea alarms may be ignored during a recruitment maneuver. Alternately, the recruitment maneuver module 214 may be in communication with alarms that are specially configured for use in a recruitment maneuver. For example, a high-pressure alarm may be set at a fixed value above a target recruitment maneuver pressure (i.e., recruitment peak inspiratory pressure, PIP) such that an alarm may sound when pressures reach a fixed pressure above the target recruitment maneuver pressure (e.g., 5 cm H₂0 above the target recruitment maneuver pressure). Other alarms and settings may be disabled or enabled during a recruitment maneuver as indicated by the care plan of each individual patient.

A pressure delivery regulator module 216 may deliver pressure into a ventilatory circuit and thereby into the lungs of a patient. By way of general overview, the basic elements impacting ventilation may be described by the following ventilatory equation:

Pₘ+Pᵥ=V/C+R*F

Here, Pₘ is a measure of muscular effort that is equivalent to the pressure generated by the muscles of a patient. If the patient's muscles are inactive, as may be the case during a recruitment maneuver, the Pₘ is equivalent to 0 cm H₂0. During inspiration, Pᵥ represents the positive pressure delivered by a ventilator (generally in cm H₂0). This ventilatory pressure, Pᵥ, represents ventilatory circuit pressure, i.e., the pressure gradient between the airway opening and the body's surface (ambient atmospheric pressure, which is set to 0 cm H₂0). This pressure gradient is what allows air to flow into the lungs of a patient. V represents the volume delivered, C refers to the respiratory compliance, R represents the respiratory resistance, and F represents the gas flow during inspiration (generally in liters per min (lpm)).

With reference to the ventilatory equation above, pressure delivery regulator module 216 may deliver pressure into a ventilatory circuit, and thereby into a patient's lungs, by any suitable method, either currently known or disclosed in the future. Further, pressure delivery regulator module 216 may regulate pressure delivery by any suitable method, either currently known or disclosed in the future. Specifically, pressure delivery regulator module 216 may be in communication with pneumatic system 102, including inspiratory module 104 coupled with inspiratory limb 132. Compressor 106 or another source(s) of pressurized gases (e.g., air, oxygen, and/or helium) may be coupled with inspiratory module 104, in communication with pressure delivery regulator module 216, to provide a gas source for delivering air pressure via inspiratory limb 132. Pressure delivery regulator module 216 may also be in communication with a pressure rate monitor module 220 and a flow monitor module 224 to adjust pressure delivery such that flow is maintained less than or equal to the peak targeted flow.

An expiratory valve regulator module 218 may regulate the release of air from the patient's lungs by regulating the size of an expiratory valve opening. By way of general overview, a ventilator initiates expiration after inspiratory flow ends by opening an expiratory valve. As such, expiration is passive, and the direction of airflow, as described above, is governed by the pressure gradient between the patient's lungs and the ambient surface pressure. Thus, the more quickly pressure is released through the expiratory valve, the correspondingly higher the resultant expiratory flow in the circuit. As the increment of pressure change escaping the patient's lungs through the expiratory valve is dependent on the size of the opening, expiratory flow may be governed by the magnitude of the opening of the expiratory valve.

Pressure rate monitor module 220 may monitor a rate of pressure change within a ventilatory circuit. As is relevant to embodiments of the present disclosure, the pressure gradient may be positive or negative, i.e., pressure may be monitored and regulated during inspiration and/or expiration. The rote of pressure change, however, may be consistently represented as a positive number, i.e., rate may be expressed as an absolute pressure change over time. Pressure rate monitor module 220 may measure or derive pressure readings itself, or alternately, may be in communication with a pressure monitor module 226, as described below. When pressure rate monitor module 220 receives pressure data from pressure monitor module 226, pressure rate monitor module 220 may then calculate the rate of pressure change over time.

During inspiration, as described above with reference to the ventilatory equation, pressure rate monitor module 220 may monitor the rate of change in pressure at the airway opening. Specifically, the pressure rate monitor module 220 may measure pressure every few milliseconds, every few seconds, or over any other suitable increment of time. Alternately, pressure rate monitor module 220 may receive pressure readings from pressure monitor module 226, as discussed above, every few milliseconds, seconds, etc. Alternately, pressure rate monitor module 220 may derive pressure data from other data and measurements included in, for example, the ventilatory equation recited above. Wherein the pressure rate monitor module 220 receives an indication from the flow monitor module 224 that the flow is greater than the peak targeted flow, the pressure rate monitor module 220 may communicate to the pressure delivery regulator module 216 to decrease pressure delivery.

During expiration, the pressure rate monitor module 220 may similarly measure a rate of pressure change. As described above for inspiration, the pressure rate monitor module 220 may again monitor the rate of change in pressure at the airway opening over any suitable increment of time. Specifically, the pressure rate monitor module 220 may measure, derive, or receive pressure data in order to monitor changes in pressure every few milliseconds, every few seconds, or over any other suitable increment of time. Wherein the pressure rate monitor module 220 receives an indication from the flow monitor module 224 that the flow is greater than the peak targeted flow, the pressure rate monitor module 220 may communicate to the expiratory valve regulator module 218 to decrease pressure release through the expiratory valve.

Monitor module 222 may include, but is not limited to, two other modules, i.e., a flow monitor module 224 and a pressure monitor module 226. Flow monitor module 224 may monitor airflow within a ventilatory circuit. Inspiratory flow may be represented as a positive flow and expiratory flow may be represented as a negative flow. However, for purposes of determining whether the flow is maintained at a low flow during either the inspiratory or the expiratory phase, the absolute value of the flow may be compared to the peak targeted flow parameter, for example 10 lpm or indeed any flow less than 10 lpm such as 9 lpm, 8 lpm, 7.5 lpm, 6 lpm, 5 lpm, 2.5 lpm or less. The choice of the peak targeted flow may be based on factors such as the anticipated time necessary to achieve a desired level of inflation of the patient's lungs, the patient's breathing period, and the rate of pressure change in the lungs. Flow may be measured or derived by any suitable method either currently known or disclosed in the future. When flow monitor module 224 determines that airflow in the ventilatory circuit is greater than peak targeted flow, flow monitor module 224 may communicate to pressure rate monitor module 220 in order to facilitate the adjustment of the rate of pressure delivered or released such that flow may be maintained less than or equal to the peak targeted flow.

In order to deliver a low flow recruitment maneuver, as disclosed by embodiments of the present methods, the rate of change in pressure, during either inspiration or expiration, may be regulated such that circuit flow is maintained less than or equal to the peak targeted flow. Specifically, during inspiration, the flow monitor module 224 may communicate with the pressure rate monitor module 220 such that the increments of pressure delivered over time result in a low flow into the lungs of a patient. In response to communications from the flow monitor module 224, the pressure rate monitor module 220 may determine a first rate of pressure change wherein the flow monitor module 224 indicates that flow is greater than peak targeted flow. The pressure rate monitor module 220 may then communicate to the pressure delivery regulator module 216 to decrease pressure delivery such that the increment of pressure change over time results in a second rate less than the first rate. Flow monitor module 224 may continually monitor flow, communicating with the pressure rate monitor module 220, which in turn may communicate with the pressure delivery regulator module 216 in order to maintain flow in the circuit less than or equal to the peak targeted flow. Alternately, the flow monitor module 224 may communicate directly with the pressure delivery regulator module 216 to adjust pressure delivery in response to a flow greater than the peak targeted flow.

The flow monitor module 224 may also maintain low flow during expiration. In this case, the flow monitor module 224 may communicate with the pressure rate monitor module 220 such that the increments of pressure released over time result in a low flow out of the lungs of a patient. In response to communications from the flow monitor module 224, the pressure rate monitor module 220 may determine a first rate of pressure change wherein the flow monitor module 224 indicates that flow is greater than peak targeted flow. The pressure rate monitor module 220 may then communicate to expiratory valve regulator module 218 to decrease pressure release by decreasing the valve opening such that an increment of pressure change over time results in a second rate less than the first rate. Flow monitor module 224 may continually monitor flow, communicating with the pressure rate monitor module 220, which may in turn communicate with the expiratory valve regulator module 218 in order to maintain flow in the circuit less than or equal to the peak targeted flow.

A pressure monitor module 226 may monitor pressure within a ventilatory circuit. Specifically, the pressure monitor module 226 may measure pressure or may derive pressure readings from other data and measurements included in, for example, the ventilatory equation recited above. Thus, pressure monitor module 226 may determine pressure as defined with reference to the pressure rate monitor module 220 above. In addition to optionally communicating pressure readings to the pressure rate monitor module 220, pressure monitor module 226 may monitor the pressure to determine when the target recruitment maneuver pressure is reached during the inspiratory phase of the recruitment maneuver. Pressure monitor module 226 may also determine when the baseline pressure is reached during the expiratory phase of the recruitment maneuver. When pressure monitor module 226 determines that either the target recruitment maneuver pressure is reached, or the baseline pressure is reached, respectively, the pressure monitor module 226 may communicate that information to the recruitment maneuver module 214.

FIG. 3 is an illustration of an embodiment of a graphical user interface for receiving clinician input for a single-breath, low flow recruitment maneuver as described herein. Specifically, FIG. 3 illustrates an embodiment of the “Recruitment Maneuver Input Screen,” as described above with reference to display module 204 and recruitment maneuver module 214.

The disclosed embodiment of the Recruitment Maneuver Input Screen 302 displays various input categories, or windows, and entry or command portals, or elements, wherein a clinician may communicate parameters and commands to the ventilator. Disclosed windows and elements may be arranged in any suitable order or configuration such that information may be communicated by the clinician to the ventilator in an efficient and orderly manner. Windows disclosed in the illustrated embodiment of the Recruitment Maneuver Input Screen 302 may be configured with elements for calling on alternate display input screens or graphical data display screens as may be provided by the ventilator. Disclosed windows and elements are not to be understood as an exclusive array, as any number of similar suitable windows and elements may be displayed for the clinician within the spirit of the present disclosure. Further, the disclosed windows and elements are not to be understood as a necessary array, as any number of the disclosed windows and elements may be appropriately replaced by other suitable windows and elements without departing from the spirit of the present disclosure. The illustrated embodiment of the Recruitment Maneuver Input Screen 302 is provided as an example of potentially useful windows and elements that may be provided to the clinician to facilitate the input of parameters and commands relevant to the disclosed delivery of a single-breath, low flow recruitment maneuver as described herein.

Further embodiments of the Recruitment Maneuver Input Screen 302 may include, for example, an optional pressure versus time window that may display how plotted data for a projected recruitment maneuver is likely to look based on the clinician input received. This optional window may appear prior to the clinician accepting the settings for the intended recruitment maneuver such that the clinician may alter various settings based on the displayed projected recruitment maneuver. When the clinician is satisfied with the displayed projected recruitment maneuver, the clinician may then accept the settings as entered and proceed with initiating the recruitment maneuver at a desired time.

In pressure input window 304, an input element 306 is provided wherein a clinician may enter a patient-specific parameter for a positive end expiratory pressure (PEEP), as will be discussed further herein. Additionally, a target recruitment maneuver pressure may be input by the clinician into element 308. The target recruitment maneuver pressure refers herein to a heightened pressure the clinician estimates may be sufficient to recruit collapsed alveoli in the lungs of a patient. Similar to a peak inspiratory pressure (PIP) associated with the general prescribed ventilation of a patient, the target recruitment maneuver (RM) pressure is the peak pressure delivered to the patient's lungs during the inspiratory phase of a single-breath recruitment maneuver, as disclosed herein. The clinician may determine the target RM pressure by any suitable means, based on any suitable patient-specific data or other suitable data, prior to entering the target RM pressure into input area of pressure input window 304. Alternately, the target RM pressure may be based on a standard formula or protocol whereby the ventilator may automatically calculate the target RM pressure or whereby the ventilator may be pre-configured with an appropriate standard target RM pressure as dictated by a hospital-specific protocol or otherwise. Where the target RM pressure is automatically calculated or preconfigured, pressure input window 304 may be configured such that an input area for the target RM pressure is inactive or not displayed to the clinician.

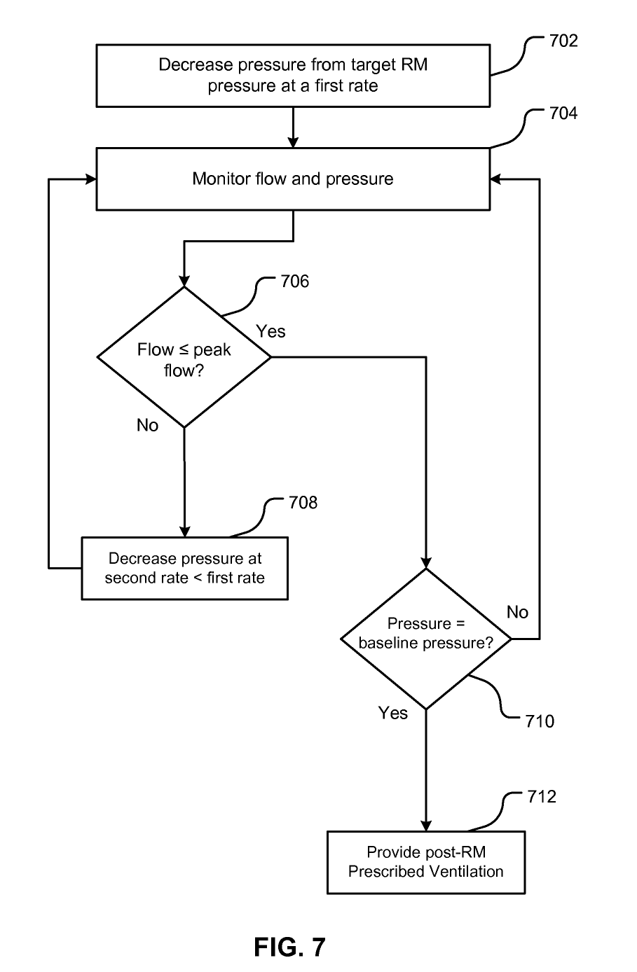

In time input window 310, an element may be provided wherein a clinician may enter a parameter for a “hold time” that the target RM pressure should be maintained in a patient's lungs. The hold time may be a manually entered time period, or may be employed as a default time period. When hold time is manually configured, the hold time may be entered as an initial setting, for instance at hold time element 312. Alternately, the hold time may be clinician-protocol specific, wherein the input screen may offer an option to select a particular clinician's protocol-specific settings among a plurality of clinician recruitment maneuver settings (not shown). In this case, the hold time may be automatically configured according to selected clinician-specific settings for the single-breath recruitment maneuver. Alternately still, the hold time may be a pre-configured default setting based on a standard formula or protocol whereby the ventilator may automatically derive an appropriate hold time or whereby the ventilator may be pre-configured with an appropriate standard hold time as dictated by a hospital-specific protocol or otherwise. Where the hold time is automatically calculated or preconfigured, time input window 310 may be configured such that an input area for the hold time is inactive or invisible to the clinician. Further still, the hold time may be set or pre-configured to a null value. For embodiments wherein the hold time is null, the methods would proceed as described herein from the end of the inspiratory phase directly to the expiratory phase of a recruitment maneuver, including but not limited to embodiments as described below in FIG. 7.

Time input window 310 may also provide an element 314 for manually configuring a “max time.” A maximum time parameter may involve a clinician-prescribed total amount of time for the completion of the single-breath recruitment maneuver. Alternately, the maximum time may be a default parameter wherein the clinician may not configure a recruitment maneuver to exceed the maximum time. The maximum time may operate to cause the ventilator to discontinue the delivery of a single-breath recruitment maneuver when the maximum time parameter is exceeded. Alternately, the ventilator may be configured to alert the clinician that the delivery of the recruitment maneuver has exceeded the maximum time parameter.

In flow input window 316, elements 318 and 320 are provided wherein a clinician may enter a parameter for a peak targeted flow for the single-breath, low flow recruitment maneuver. The peak targeted flow may be set for both inspiration, at element 318, and expiration, at element 320; or for either phase individually to the exclusion of the other. Alternately still, the clinician may not want to configure either phase of the recruitment maneuver for low flow. The peak targeted flow, similar to a flow threshold, may enable the ventilator to regulate flow such that a recruitment maneuver may be delivered at low flow. Generally, if peak targeted flow is breached, the ventilator may adjust pressure and/or other suitable ventilatory parameters to reduce flow below peak targeted flow (such that the recruitment maneuver may be delivered at low flow). Additionally, an alert may advise the clinician that flow was detected above the peak targeted flow and, as such, that pressure-volume data may have been affected. That is, in cases where the recruitment maneuver was not delivered at low flow, a pressure drop due to resistance may have been significant, decreasing the value of the pressure-volume data collected during the recruitment maneuver.

According to embodiments, as with reference to the parameter settings above, the peak targeted flow (for either inspiration or expiration) may be a manually entered or may be pre-configured as a default peak targeted flow. When peak targeted flow is manually configured, the clinician may determine an appropriate peak targeted flow based on any acceptable clinical or other patient-specific parameters or data. Alternately, peak targeted flow may be clinician-protocol specific, wherein the input screen may offer an option to select a particular clinician's protocol-specific settings among a plurality of clinician recruitment maneuver settings (not shown). In this case, the peak targeted flow may be automatically configured according to selected clinician-specific settings for the single-breath recruitment maneuver. Alternately still, the peak targeted flow may be a pre-configured default setting based on a standard formula or protocol whereby the ventilator may automatically derive an appropriate peak targeted flow or whereby the ventilator may be pre-configured with an appropriate standard peak targeted flow as dictated by a hospital-specific protocol or otherwise, for instance less than or equal to 10 liters per minute (lpm). Where the peak targeted flow is automatically calculated or preconfigured, flow input window 316 may be configured such that an input area for the peak targeted flow is inactive or invisible to the clinician.

An optional settings input window 322 may provide elements wherein a clinician may enter any number of optional parameters and settings. Optional settings input window 322 is termed optional not to imply in any sense that inputs into this window are unnecessary or unimportant. Rather, settings entered into this window are not directly related to the delivery of a single-breath recruitment maneuver as described herein, but rather may be more applicable to the prescribed ventilation of a patient, and thus, may be entered elsewhere in the configurations settings and screens available to the clinician on the ventilator. However, it may be that one or more parameters are of interest to a particular clinician, or to a standard or hospital protocol as described above. As such, the optional settings input window 322 is provided to allow for input of additional ancillary parameters for monitor or display during the single-breath recruitment maneuver. For instance, a value for fractional inspiratory oxygen (FiO₂) may be input into element 324 by the clinician for monitor and control during the recruitment maneuver. As described previously, the clinician may determine an appropriate FiO₂ input by any acceptable clinical or other patient-specific parameters or data. Optional settings input window 322 may also provide trigger controls whereby the clinician may call on other appropriate settings screens or displays, for instance stored recruitment maneuver settings at element 326, including: one or more previously saved recruitment maneuver settings for a particular patient; one or more selections for clinician-specific recruitment maneuver settings; or one or more selections for standard recruitment maneuver settings based on any appropriate standard or hospital-specific protocol. Optional settings input window 322 may also provide for configuring any customized settings not previously discussed or disclosed, but desired by a particular clinician and available on other settings screens or displays of the ventilator (not shown). As such, a customize trigger control may provide for calling on alternate displays and drawing particular parameter settings into the Recruitment Maneuver Input Screen, as desired by a clinician.

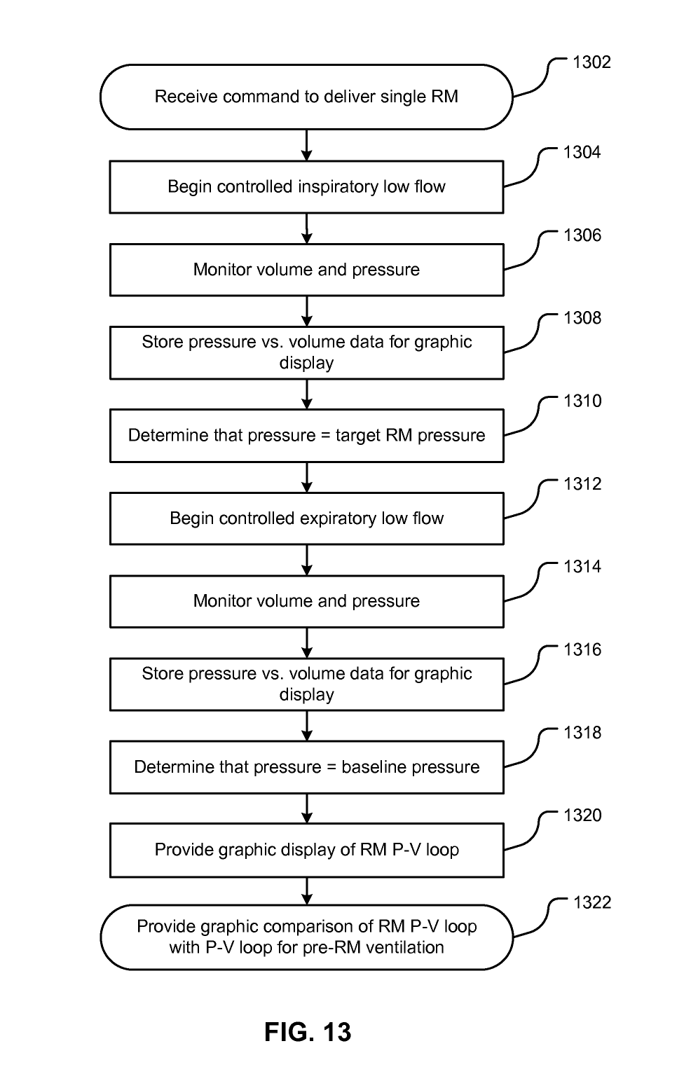

A graphics input window 328 may provide elements wherein a clinician may select any number of graphical representations of recruitment maneuver data to be calculated and displayed. Specifically, the clinician may select a graphical display of a volume curve (not shown), a pressure curve at element 332, a flow curve at element 330, etc., for the recruitment maneuver. Additionally, the clinician may desire to compare any of these recruitment maneuver curves with curves calculated during prescribed ventilation of a patient, either before or after the delivery of the recruitment maneuver (not shown). The clinician may also make a selection at element 334 to display a recruitment maneuver pressure-volume loop (PV loop). The details of the generation and display of one or more PV loops will be discussed further herein with reference to FIG. 13. The clinician may also select a comparison display of a PV loop generated from the single-breath recruitment maneuver against pre- or post-recruitment maneuver PV loops generated from data during prescribed ventilation at element 336. The Recruitment Maneuver Input Screen 302 may be further configured to offer other suitable and useful graphical display representations of the recruitment maneuver data, as well as any other suitable comparison display that may be useful to the clinician.

A prescribed ventilation resume window 338 may also be provided wherein a clinician may configure the post-recruitment maneuver prescribed ventilation settings. The clinician may select post-recruitment maneuver prescribed ventilation to be resumed according to pre-recruitment maneuver settings by selecting a “Y” control at element 340, or any other suitable command. Alternately, the clinician may manually configure the post-recruitment maneuver prescribed ventilation by entering parameters into element 342. In the embodiment shown, selection of “Y” may result in the generation of a secondary window (not shown) that allows input of the desired settings. As discussed previously for other parameters, the clinician may determine and configure appropriate post-recruitment maneuver prescribed ventilation settings based on any acceptable clinical or other patient-specific parameters or data. Further, control selections in the prescribed ventilation resume window 338 may provide for saving the single-breath recruitment maneuver settings upon completion of the recruitment maneuver at element 344.

Although currently the delivery of recruitment maneuvers may demand almost complete sedation or paralysis of a patient, low flow recruitment may presently, or in the future, allow for spontaneously breathing patients to remain conscious during a recruitment maneuver. As such, an element 346 may be available in settings upon prescribed ventilation resume window 338 to synchronize the end of the recruitment maneuver with the beginning of prescribed ventilatory inspiration in cases of a spontaneously breathing patient. At the prescribed ventilation resume window 338, or alternately at optional settings input window 322, elements for initiating a single-breath recruitment maneuver, i.e., element 348, and for interrupting a single-breath recruitment maneuver, i.e., element 350, may be provided. Additionally, settings upon prescribed ventilation resume window 338 may be configured to offer the clinician any other suitable selection or command controls relevant to resuming the prescribed ventilation of the patient.

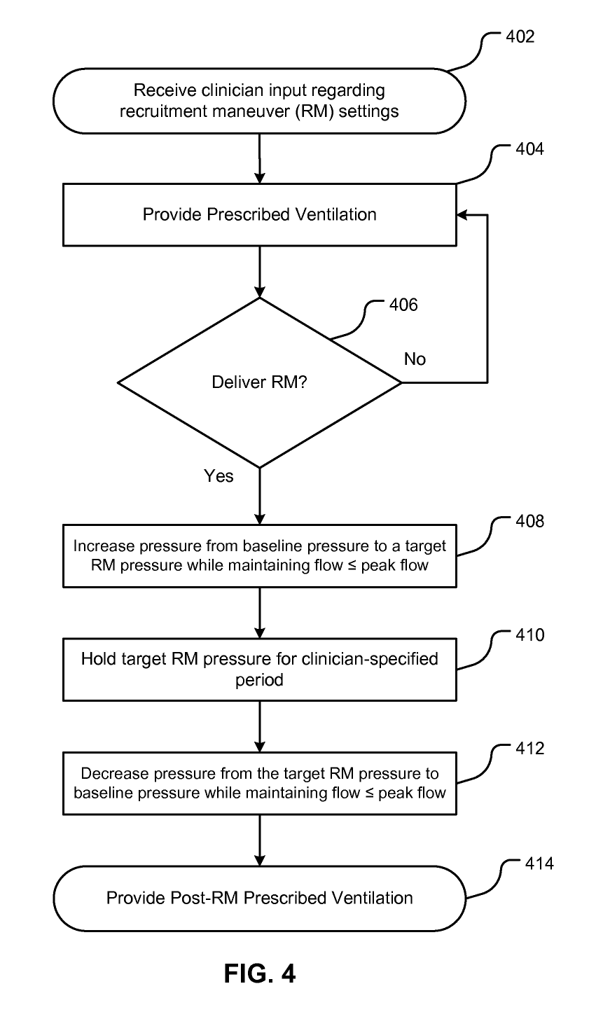

FIG. 4 is a flow-diagram illustrating embodiments of a method for delivering a single-breath, low flow recruitment maneuver as described herein. At receive clinician input operation 402, the ventilator may receive settings, as described above, for delivering a single-breath recruitment maneuver. Clinician input may be received at any suitable time prior to initiating the recruitment maneuver, i.e., it may be set during the initial ventilator set-up, or any suitable time during provide prescribed ventilation operation 404. As has been described previously with reference to FIG. 3, the clinician may configure recruitment maneuver settings by calling on a single-breath recruitment maneuver input screen, for example, from a main ventilation input display.

At a deliver RM determination operation 406, it is determined whether or not to deliver the recruitment maneuver. This decision may be based upon an indication from the clinician at initial set-up, i.e., by receiving input to initiate the recruitment maneuver at a particular time or at an increment of time after initiation of the provide prescribed ventilation operation 404. The clinician may further configure the ventilator to deliver recruitment maneuvers on a set schedule, having an interval of time between each recruitment maneuver. Deliver RM determination operation 406 may also be based on a clinician “Initiate Recruitment Maneuver” command, as described above.

Ventilators are configured to mimic natural breathing, exhibiting inspiration and expiration. During inspiration the ventilator delivers oxygenated air to the patient's lungs and during expiration oxygen-depleted (carbon dioxide rich) air is returned to the ventilator. Thus, the graphic representation of the above ventilatory formula is expressed in wave form. A particular breath may be further divided into four phases, including: transition from expiration to inspiration (triggering), inspiration, transition from inspiration to expiration (cycling), and expiration.

The pressure from which a ventilator initiates inspiration is termed the “baseline” pressure. This pressure may be atmospheric pressure (0 cm H₂0), also called zero end-expiratory pressure (ZEEP). Alternately, the baseline pressure may be positive, termed positive end-expiratory pressure (PEEP).

At an increase pressure operation 408, the recruitment maneuver begins increasing pressure from a baseline pressure to a target recruitment maneuver pressure. As pressure is being delivered to the patient's lungs, this increase in pressure represents an inspiratory portion of the recruitment maneuver. As will be discussed in more detail below, the inspiratory pressure may be delivered at a constant or a variable rate (i.e., change in pressure over time) such that the flow remains less than or equal to a peak targeted flow. The peak targeted flow may be entered as an initial setting in the Recruitment Maneuver Input screen. Alternately, the peak targeted flow may represent a default setting that is recruitment maneuver protocol-specific for a particular hospital, a particular clinician, etc. Embodiments of the present methods may configure the peak targeted flow at less than or equal to 10 lpm, for example, as set by either the clinician or as a default setting.

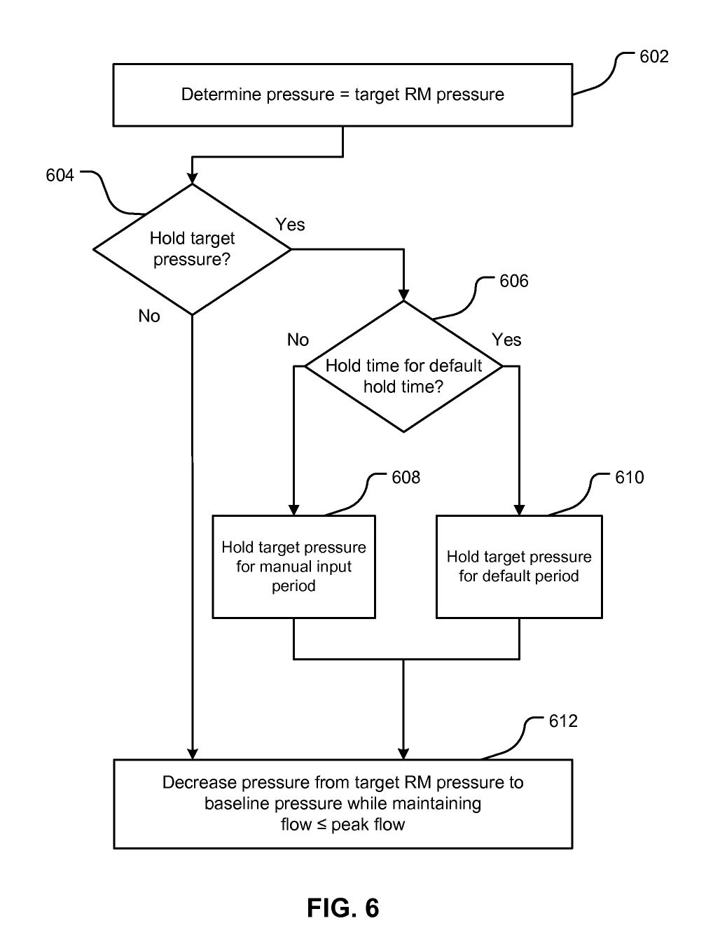

At a hold operation 410, the ventilator may maintain the target recruitment maneuver pressure for a period of time, termed herein a “hold time.” As has been described above with reference to FIG. 3, and as will be discussed further herein with reference to FIG. 6, the hold time may be any suitable amount of time, or no amount of time. During hold operation 410, when appropriate, the ventilator may maintain the target recruitment maneuver pressure for a clinician-specified or a default hold time. When the hold time is set to a null value, indicating that the target recruitment maneuver pressure should not be held for any period of time, the methods may progress directly to decrease pressure operation 412.

At the decrease pressure operation 412, the ventilator may begin the expiratory phase of the recruitment maneuver. In general, expiration is technically the period between inspirations. A ventilator initiates the expiratory phase after inspiratory flow ends by opening an expiratory valve. As expiration is passive, the expiratory flow in the ventilatory circuit may be governed by the magnitude of the opening of the expiratory valve over time. Embodiments of the present methods may decrease pressure from the target recruitment maneuver pressure back to the baseline pressure while maintaining the flow less than or equal to a peak targeted flow. As will be discussed further below, the flow may be monitored by the ventilator such that the ventilator may adjust the release of pressure through the expiratory valve to maintain the flow less than or equal to the desired peak targeted flow.

At a post-RM prescribed ventilation operation 414, the ventilator may resume prescribed ventilation of a patient. The prescribed ventilation may be according to the pre-recruitment maneuver ventilatory settings of the patient. Alternately, the clinician may input special ventilatory settings that may be initiated after the recruitment maneuver. The clinician may set post-recruitment maneuver ventilatory settings during the initial ventilator setup, or at any suitable time during ventilation prior to the delivery of post-recruitment maneuver prescribed ventilation. Embodiments, described in greater detail below, may also allow the ventilator to synchronize the end of a recruitment maneuver with the initiation of post-recruitment maneuver prescribed ventilation.

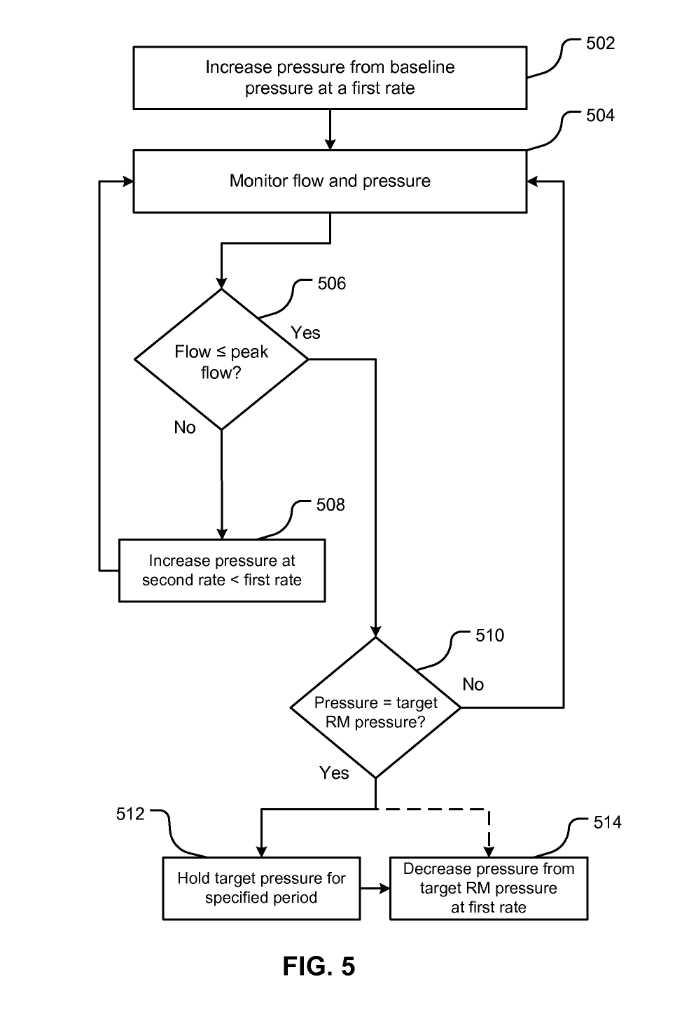

FIG. 5 is a flow-diagram illustrating an embodiment of a method for delivering an inspiratory portion of a single-breath, low flow recruitment maneuver as described herein. Specifically, FIG. 5 illustrates an embodiment of the increase pressure operation 408, as described above in FIG. 4, wherein the ventilator may increase pressure from a baseline pressure to a target recruitment maneuver pressure.

At a first increase pressure operation 502, the ventilator may begin by increasing pressure in the ventilatory circuit at a first rate. At a monitor operation 504, the ventilator may monitor flow and/or pressure. Specifically, as described above, flow may be derived according to the following equation: F=V/time increment (wherein the change in volume is known). Alternately, the flow may be measured at selected points along the ventilatory circuit, as describe herein with reference to flow monitor module 224. Thus, monitoring flow may involve any suitable method for measuring or deriving flow within the ventilatory circuit.

In addition, during monitor operation 504, the ventilator may monitor the pressure in the ventilatory circuit to determine if the target RM pressure has been reached. Monitor operation 504 may also monitor a rate of pressure change, as described above with reference to the pressure rate monitor module 220.

At a flow determination operation 506, the ventilator may determine by any suitable method, including but not limited to methods described above, whether the circuit flow, as measured or derived, is less than or equal to the peak targeted flow. If it is determined that the flow is greater than the peak targeted flow, the process may progress to pressure determination operation 510. If it is determined that the flow is not less than or equal to the peak targeted flow, the process may progress to a second increase pressure operation 508.

During the second increase pressure operation 508, the ventilator may adjust pressure delivery such that the pressure is increased at a second rate that is less than the first pressure-delivery rate. Embodiments may then return to the monitor operation 504. During monitor operation 504, embodiments of the methods described herein may continue to monitor circuit flow and pressure by any suitable method. The methods may again continue, as previously described, from monitor operation 504 to flow determination operation 506.

At a pressure determination operation 510, the ventilator may determine by any suitable method, including but not limited to methods described above, whether the pressure, as measured or derived, is about equal to the target recruitment maneuver pressure. Embodiments of the present methods envision determination methods in which the pressure is monitored at sufficiently small intervals such that when the pressure reaches the target recruitment maneuver pressure the system is able to make such a determination within an acceptable degree of error. As such, “about” refers to the acceptable degree of error by which the system may make a pressure determination. When it is determined that the pressure is not about equal to the target recruitment maneuver pressure, the process may return to monitor operation 504. At monitor operation 504, embodiments of the methods described herein may continue to monitor circuit flow and pressure by any suitable method. The methods may again continue, as previously described, from monitor operation 504 to flow determination operation 506. When it is determined that the pressure is about equal to the target recruitment maneuver pressure, the process may progress to hold pressure operation 512 or, alternately, to decrease pressure operation 514.

At hold pressure operation 512, after pressure determination operation 510 determines that the pressure is equivalent to or greater than the target recruitment maneuver pressure, embodiments of the present methods may maintain the target recruitment maneuver pressure for a hold time, as described above at hold operation 410. Alternately, embodiments of the present methods may progress directly to decrease pressure operation 514, wherein the expiratory phase of the recruitment maneuver may be initiated, as described at decrease pressure operation 412, and as described further with reference to FIG. 7.

FIG. 6 is a flow-diagram illustrating an embodiment of a method for delivering a single-breath, low flow recruitment maneuver incorporating a hold time as described herein. Specifically, FIG. 6 illustrates an embodiment of hold operation 410, as described above with reference to FIG. 4, wherein the ventilator may maintain the target recruitment maneuver pressure in the lungs for a hold time.

At a pressure determination operation 602, the ventilator may determine by any suitable method, including but not limited to methods described above with reference to pressure determination operation 510, that the pressure, as measured or derived, is about equal to the target recruitment maneuver pressure.

At a hold determination operation 604, the ventilator may determine whether the target recruitment maneuver pressure should be maintained in the lungs for a hold time according to the settings for the recruitment maneuver. As discussed above with reference to the Recruitment Maneuver Input Screen illustrated in FIG. 3, a hold time may be a manually entered time period, or may be employed as a default hold time period. Alternately, some disclosed embodiments may not require a hold time, and in such case FIG. 6 is inapplicable and the methods would proceed as described for the expiratory phase of a recruitment maneuver, including but not limited to embodiments as described below in FIG. 7.

At default hold determination operation 606, the ventilator may determine whether the hold time should be employed as a default hold time. In this case, the hold time may be configured according to a recruitment maneuver protocol for a particular hospital, or any other suitable default hold time. When the ventilator determines that the hold time is a default hold time, embodiments of the present methods may progress to a default hold operation 610. Alternately, when the ventilator determines that the hold time is a not a default hold time, embodiments of the present methods may progress to manual hold operation 608.

At manual hold operation 608, when the ventilator determines that the hold time is a not a default hold time, the ventilator may pause for a clinician-specified hold time in which the target recruitment maneuver pressure is maintained in the lungs for the clinician-specified hold period. According to disclosed embodiments, this clinician-specified hold period begins after inspiratory flow has ended, but before opening the expiratory valve. After maintaining the target recruitment maneuver pressure in the lungs for the clinician-specified period, the methods may progress to a decrease pressure operation 612.

At the default hold operation 610, the ventilator may pause for a default hold time in which the target recruitment maneuver pressure is maintained in the lungs for the default period. According to disclosed embodiments, this default hold period begins after inspiratory flow has ended, but before opening the expiratory valve. After maintaining the target recruitment maneuver pressure in the lungs for the default period, the methods may progress to the decrease pressure operation 612.

At the decrease pressure operation 612, the ventilator may begin decreasing pressure in the ventilatory circuit from the target recruitment maneuver pressure to the baseline pressure while maintaining the flow less than or equal to the peak targeted flow, as described above.

FIG. 7 is a flow-diagram illustrating an embodiment of a method for releasing an expiratory phase of a single-breath, low flow recruitment maneuver as described herein. Specifically, FIG. 7 illustrates an embodiment of decrease pressure operation 412, as described above with reference to FIG. 4, wherein the ventilator may decrease pressure from the target recruitment maneuver pressure to a baseline pressure.

At a first decrease pressure operation 702, the ventilator may begin decreasing pressure in the ventilatory circuit at a first rate, including but not limited to the methods disclosed for expiratory valve regulator module 218 and pressure rate monitor module 220. The ventilator may monitor flow and/or pressure at monitor operation 704. Flow may be monitored according to any suitable method for measuring or deriving flow within the ventilatory circuit, including but not limited to the methods described above for flow monitor module 224.

In addition to monitoring flow at monitor operation 704, the ventilator may also monitor pressure. Again, pressure may be may be monitored according to any suitable method for measuring or deriving pressure within the ventilatory circuit, including but not limited to the methods described above with reference to pressure monitor module 226 and pressure rate monitor module 220.

At a flow determination operation 706, the ventilator may determine by any suitable method, including but not limited to methods described above, whether the circuit flow is less than or equal to the peak targeted flow. If it is determined that the flow is not less than or equal to the peak targeted flow, the process may progress to a second pressure decrease operation 708. If it is determined that the flow is less than or equal to peak targeted flow, the process may progress to pressure determination operation 710.

At the second pressure decrease operation 708, the ventilator may adjust pressure release such that the pressure is decreased at a second rate that is less than the first pressure-release rate. Embodiments may then return to monitor operation 704. At monitor operation 704, embodiments of the methods described herein may continue to monitor circuit flow and pressure by any suitable method. The methods may again continue, as previously described, from monitor operation 704 to flow determination operation 706, and so on.

At pressure determination operation 710, after determining that the flow is less than or equal to the peak targeted flow, the ventilator may determine by any suitable method, including but not limited to methods described above, whether the pressure is about equal to a baseline pressure. When it is determined that the pressure is not about equal to the baseline pressure, the process may return to monitor operation 704. At monitor operation 704, embodiments of the methods described herein may continue to monitor circuit flow and pressure by any suitable method. The methods may again continue, as previously described, from monitor operation 704 to flow determination operation 706, and so on. When it is determined that the pressure is about equal to the baseline pressure, the process may progress to post-RM prescribed ventilation operation 712.

At post-RM prescribed ventilation operation 712, after determining at pressure determination operation 710 that the pressure is about equivalent to the baseline pressure, embodiments of the present methods may begin to provide prescribed ventilation, as described above at post-RM prescribed ventilation operation 414.

It will be clear that the systems and methods described herein for a single-breath low flow recruitment maneuver are well adapted to attain the ends and advantages mentioned as well as those inherent therein. Those skilled in the art will recognize that the methods and systems within this specification may be implemented in many manners and as such is not to be limited by the foregoing exemplified embodiments and examples. In other words, functional elements being performed by a single or multiple components, in various combinations of hardware and software, and individual functions can be distributed among software applications at either the client or server level. In this regard, any number of the features of the different embodiments described herein may be combined into one single embodiment and alternate embodiments having fewer than or more than all of the features herein described are possible.

While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the appended claims.

### Multi-Breath Recruitment Maneuvers

Multi-breath recruitment maneuvers may also be employed to expand collapsed alveoli in order to increase the functional surface area of a patient's lungs. The benefits of providing recruitment maneuvers in a multi-breath series include, among others, the increased potential for recruitment maneuvers to be delivered to patients while conscious. In addition to the single breath methods described above, multi-breath maneuvers may offer additional flexibility for tailoring maneuvers to the needs of a particular patient. For instance, multi-breath maneuvers may be employed by incrementally increasing the PEEP, or baseline pressure, rather than the PIP, as with the target recruitment maneuver pressure described above. Alternately, multi-breath maneuvers may provide any of the functionalities previously discussed for single-breath maneuvers. To facilitate the disclosure of the methods and modules herein, embodiments of multi-breath recruitment maneuvers will be discussed, and may include any suitable variation on the following descriptions.

An embodiment of a multi-breath recruitment maneuver may involve maintaining the prescribed PIP (peak inspiratory pressure, as described further below) and increasing the PEEP (positive-end expiratory pressure, as described further below) over the duration of the multi-breath recruitment maneuver. For example, the multi-breath recruitment maneuver may be configured with an ascending phase and a descending phase. The ascending phase may involve, for instance, 10 breaths per ascending breath set. The multi-breath recruitment maneuver may be further configured, for example, to have 6 ascending breath sets. The PIP, for example, may be held constant for each breath within each ascending breath set. The PEEP may be configured to increase by one increment after each ascending breath set through the end of the final ascending breath set, i.e., increasing to a peak PEEP value during the sixth ascending breath set. A descending phase of the multi-breath recruitment maneuver may then be initiated and configured to step the PEEP back down to the initial baseline pressure, either over a single breath, over a plurality of breaths, or over any number of breath sets. In one embodiment, the descending phase may mirror the steps of the ascending phase upward with equivalent steps, or sets, downward.

Another embodiment of a multi-breath recruitment maneuver may involve incrementally increasing the PIP to a target recruitment maneuver pressure. The PIP may increase incrementally for each ascending breath set, as described above for the PEEP, wherein the clinician may designate a total increase in PIP over the maneuver and the ventilator may calculate the incremental increase for each ascending breath set. One embodiment may further disclose incrementally increasing both the PEEP and the PIP for each breath set. Alternately, another embodiment may only incrementally increase the PIP while maintaining the PEEP at the initial baseline level. Again, as described above, a descending phase may involve a stepwise decrease of the PIP, and if necessary, the PEEP. As described above, the descending phase may involve a single breath, a plurality of breaths, or any suitable number of breath sets.

In sum, PEEP and/or PIP may be increased or decreased an aggregate amount over the ascending and/or descending phase of a multi-breath recruitment maneuver. Further, the ascending and/or descending phase may be divided into a number of ascending and/or descending breath sets (xₙ), such that the incremental increase or decrease in PEEP or PIP for each breath set is equal to the total change in PEEP divided by the total number of breath sets. Alternately, the clinician may input an incremental increase or decrease in PEEP or PIP by selecting a pre-determined increment or manually inputting an increment, rendering a calculation of the increment unnecessary. Alternately still, the ascending and/or descending phase may be divided into a number of breaths, such that the incremental increase or decrease in PEEP or PIP for each breath is equal to the total change in PEEP divided by the total number of breaths. Again, rather than by calculation, the clinician may input an incremental increase and/or decrease for each breath, either by manual input or selection, or as a pre-configured setting as determined by machine protocol settings. In the illustrated embodiment, each breath within an ascending or descending breath set shares the same incremental change in PEEP and/or PIP. However, the incremental change may be configured to vary from breath set to breath set or from breath to breath, as dictated by the care plan of a particular patient and the desires of a clinician.

In another embodiment of a multi-breath recruitment maneuver, each breath of the multi-breath recruitment maneuver may be configured to attain a target recruitment maneuver pressure, wherein the target recruitment maneuver pressure may be any suitable pressure greater than the prescribed PIP. In this case, the multi-breath maneuver may be likened to a series of single-breath maneuvers, as described above. An embodiment may further involve incrementally increasing the PEEP, according to any suitable method as described above or as otherwise appropriate. In this case, the multi-breath maneuver would depart from the above disclosure regarding single-breath maneuvers. In either case, descending phases may involve a stepwise decrease from the target recruitment maneuver pressure and, if necessary, the PEEP as well. As described above, the descending phase may involve a single breath, a plurality of breaths, or any suitable number of descending breath sets.

The reader will understand that any combination of the methods disclosed above for alternate embodiments of a multi-breath, low flow recruitment maneuver may result in further embodiments that are well within the spirit of the disclosed methods and systems.

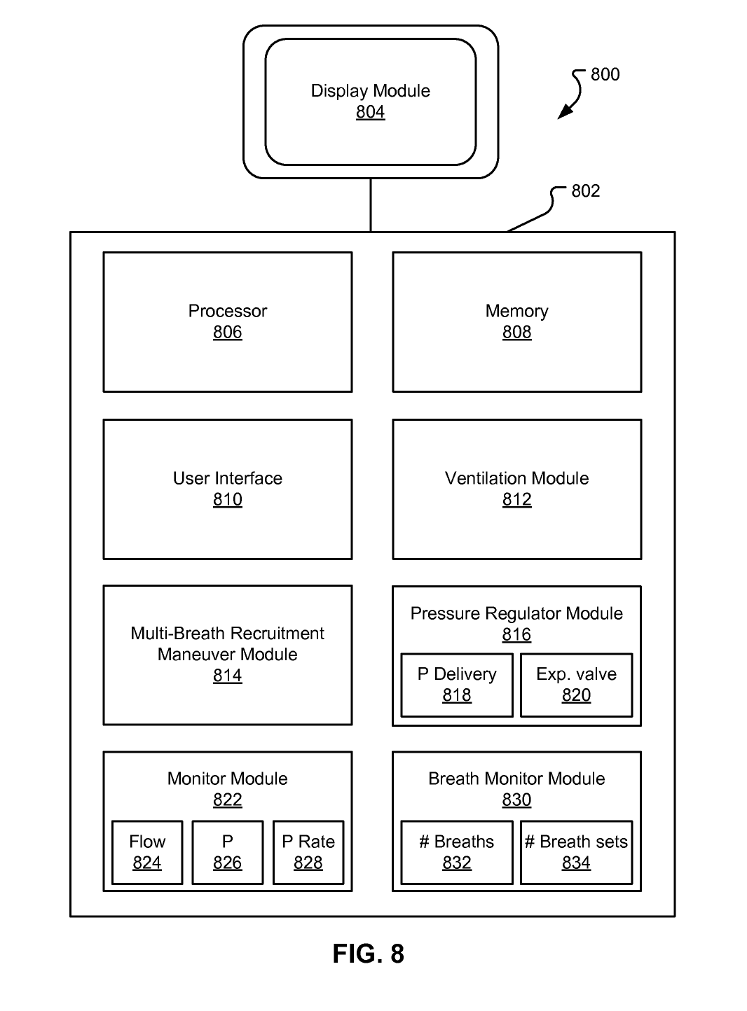

FIG. 8 is a block-diagram illustrating an embodiment of a ventilatory system 800 for delivering a multi-breath, low flow recruitment maneuver as described herein.

The ventilator 802 includes various ventilation and recruitment maneuver modules 812-834, a display module 804, memory 808, one or more processors 806, and user interface 810. Memory 808 is defined as described above for memory 112 and memory 208. Similarly, the one or more processors 806 are defined as described above for the one or more processors 116 and 206. Processors 806, as with processors 206, may further be configured with a clock whereby elapsed time may be monitored by the system 800.

The display module 804 displays various input screens to a clinician, including but not limited to a Multi-Breath RM Input Screen, as described further herein, for receiving clinician input. The display module 804 is configured to communicate with user interface 810, and is further defined as described above for display module 204 and user interface 210.

Ventilation module 812 oversees prescribed ventilation as delivered to a patient, and is further defined as described above for ventilation module 212.

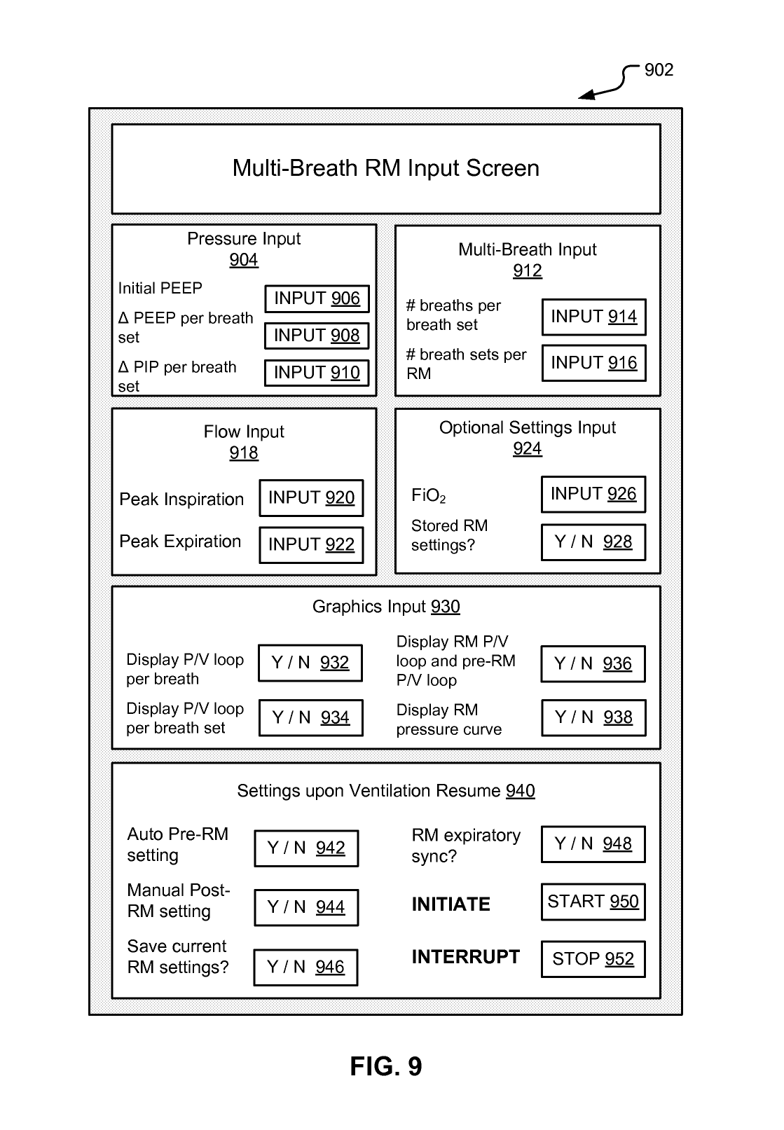

Multi-breath recruitment maneuver module 814 oversees multi-breath recruitment maneuvers as delivered to a patient, and is further defined as described above for recruitment maneuver module 214. As will be described further herein with reference to FIG. 9, the clinician may configure multi-breath recruitment maneuver settings by calling on a Multi-Breath RM Input Screen from a main ventilation input display menu. Input for a multi-breath recruitment maneuver may include, but is not limited to, input regarding a target recruitment maneuver pressure, or alternately, a peak inspiratory pressure (PIP). Input may further include settings for an initial PEEP and, optionally, an incremental or total increase in PEEP over the full multi-breath maneuver. Further still, the clinician may set a number of breaths per a multi-breath set in the maneuver, as well as a total number of multi-breath sets in the maneuver. The multi-breath sets may be configured for an ascending phase of a multi-breath recruitment maneuver and additionally or optionally, for a descending phase of a multi-breath recruitment maneuver. Alternately, the clinician may desire the descending phase to occur over a single breath (returning to the initial PEEP setting in one breath), or over a total number of breaths (but not divided into multiple sets of breaths). Additionally, a hold time for the recruitment maneuver target pressure, or a peak inspiratory pressure, for each breath within the multi-breath maneuver may be configured. As well, a peak targeted flow setting and settings for delivering post-recruitment maneuver prescribed ventilation may be entered. Clinician input may be received at any suitable time during or before the prescribed ventilation and before initiating the multi-breath recruitment maneuver.

Specifically, the multi-breath recruitment maneuver module 814 may be in communication with the various recruitment maneuver modules described below to oversee the completed delivery of the multi-breath recruitment maneuver. For example, the multi-breath recruitment maneuver module 814 may initiate the first inspiratory breath of the first inspiratory breath set for a multi-breath, low flow recruitment maneuver; transition from one phase of the recruitment maneuver to the next (e.g., transitioning from a first breath set to the next breath set, etc.); and synchronize the transition back to the prescribed ventilation of a patient—in addition to the features described above with reference to recruitment maneuver module 214. The multi-breath recruitment maneuver module 814 may also orchestrate and supervise any number of other functions in order to provide a multi-breath recruitment maneuver to a patient.

Pressure regulator module 816 may include, but is not limited to, two other modules, i.e., pressure delivery module 818 and expiratory valve regulator module 820. Pressure regulator module 816 is responsible for regulating pressure within the ventilatory circuit, both during inspiration and expiration. Thus, the pressure regulator module 816 may be in communication with a monitor module 822 in order to adjust the delivery and/or release of air pressure to ensure low flow during the multi-breath recruitment maneuver. Pressure delivery regulator module 818 is defined as described above for pressure delivery regulator module 216. Expiratory valve regulator module 820 is defined as described above for expiratory valve regulator module 218.

Monitor module 822 may include, but is not limited to, three other modules, i.e., flow monitor module 824, pressure monitor module 826, and pressure rate monitor module 828. Flow monitor module 824 may monitor airflow within a ventilatory circuit and is defined as described above for flow monitor module 224. Pressure rate monitor module 828 may monitor a rate at which pressure changes occur within ventilatory circuit and is defined as described above for pressure rate monitor module 220.

Pressure monitor module 826 may include additional responsibilities during a multi-breath recruitment maneuver. Pressure monitor module 826 may perform the functions described above with reference to pressure monitor module 226. Additionally, pressure monitor module 826 may be responsible for monitoring the potentially variable PEEP, PIP, and/or target recruitment maneuver pressure parameters during delivery of a multi-breath recruitment maneuver, as described specifically below regarding various embodiments of the disclosed methods. The pressure monitor module 826 may then communicate with the multi-breath recruitment maneuver module 814 regarding any of the above parameters (PEEP, PIP, etc.), or any other suitable parameters, according to the settings for a particular multi-breath recruitment maneuver.

Breath monitor module 830 may, but is not limited or required to, include two other modules, i.e., number breaths module 832 and number breath sets module 834. The number breath sets module 834 may receive input parameters, for example from the user interface 810, for a number of breath sets in an ascending phase of a multi-breath recruitment maneuver and in a descending phase of a multi-breath recruitment maneuver. Further, the number breaths module 832 may receive input parameters regarding the number of breaths that are included in each ascending or descending breath set and/or a total number of breaths in the recruitment maneuver. The breath monitor module 830, alone or in combination with the other modules described, may monitor the number of breaths by any suitable means. The breath monitor module 830 may then determine when each breath set has occurred and may communicate that information to the multi-breath recruitment maneuver module 814, or other suitable modules. Alternately, if the multi-breath low flow recruitment maneuver is configured without breath sets, the breath monitor module 830 may rather monitor the total number of elapsed breaths and communicate that information to the multi-breath recruitment maneuver module 814, or other suitable modules.

FIG. 9 is an illustration of an embodiment of a graphical user interface for receiving clinician input for a multi-breath, low flow recruitment maneuver as described herein. Specifically, FIG. 9 illustrates an embodiment of the Multi-Breath RM Input Screen, as described above with reference to display module 804 and multi-breath recruitment maneuver module 814.

The disclosed embodiment of the Multi-Breath RM Input Screen 902 displays various input categories, or windows, and entry or command portals, or elements, wherein a clinician may communicate parameters and commands to the ventilator. Disclosed windows and elements may be arranged in any suitable order or configuration such that information may be communicated by the clinician through the controls to the ventilator in an efficient and orderly manner. Elements disclosed in the illustrated embodiment of the Multi-Breath RM Input Screen 902 may be configured to call on alternate display input screens or graphical data display screens as may be provided by the ventilator. Disclosed windows and elements are not to be understood as an exclusive array, as any number of similar suitable windows and elements may be displayed for the clinician within the spirit of the present disclosure. Further, the disclosed windows and elements are not to be understood as a necessary array, as any number of the disclosed windows and elements may be appropriately replaced by other suitable windows and elements without departing from the spirit of the present disclosure. The illustrated embodiment of the Multi-Breath RM Input Screen 902 is provided as an example of potentially useful windows and elements that may be provided to the clinician to facilitate the input of parameters and commands relevant to the disclosed delivery of a multi-breath, low flow recruitment maneuver as described herein.

Further embodiments of the Multi-Breath RM Input Screen 902 may include, for example, an optional pressure versus time window that may display how plotted data for a projected recruitment maneuver is likely to look based on the clinician input received. This optional window may appear prior to the clinician accepting the settings for the intended recruitment maneuver such that the clinician may alter various settings based on the displayed projected recruitment maneuver. When the clinician is satisfied with the displayed projected recruitment maneuver, the clinician may then accept the settings as entered and proceed with initiating the recruitment maneuver at a desired time.

A pressure input window 904 may be provided, wherein an element is provided for entering a patient-specific parameter for an initial positive end expiratory pressure (PEEP). The initial PEEP may be the PEEP associated with the prescribed ventilation of a patient, or alternately, an initial PEEP may be specially selected for the multi-breath recruitment maneuver. Additionally, an increase in PEEP for each breath set may be input. Alternately, a total PEEP increase may be entered (not shown) (in which case, for a selected number of breath sets, PEEP may be increased for each breath set by an increment equal to the total PEEP increase divided by the selected number of breath sets). If the clinician chooses not to configure the multi-breath recruitment maneuver with a number of breath sets, a total PEEP increase may be entered (not shown) (in which case, each breath may increase PEEP by an increment equal to the total PEEP increase divided by the total number of breaths).

In pressure input window 904, a change in peak inspiratory pressure (PIP) (the PIP being associated with the prescribed ventilation of the patient) may be input by the clinician for each breath set. In this case, the total increase in PIP over the multi-breath recruitment maneuver may be input (not shown) and the ventilator may then calculate an incremental increase in PIP for each breath set by dividing the total PIP by the number of breath sets. Alternately, the incremental increase per breath set may be input by a clinician directly at element 908. If the clinician chooses not to configure the multi-breath recruitment maneuver with a number of breath sets, an absolute increase in PIP may be entered (i.e., a target recruitment maneuver pressure, not shown) (in which case, each breath may increase PIP by an increment equal to the total PIP increase divided by the total number of breaths). The clinician may determine appropriate inputs for the initial PEEP into element 906, change in PEEP into element 906, total PIP (not shown), and change in PIP into element 910, by any suitable means, based on any suitable patient-specific data or other suitable data. Note further that the above input elements may not fully address all embodiments disclosed or inherent for the delivery of a multi-breath recruitment maneuver. For instance, if the multi-breath, low flow recruitment maneuver is configured such that each breath of the multi-breath recruitment maneuver is delivered to reach a target recruitment maneuver pressure, the change in pressure may be attained by each breath and may not be divided into increments over a number of breaths, as described above. Various other calculations and input parameters may be appropriate for different embodiments of delivering a multi-breath low flow recruitment maneuver and the discussion above is not meant to limit the present methods to the disclosed calculations and input parameters discussed above.

In a multi-breath input window 906, an element 914 is provided wherein a clinician may enter a number of breaths per breath set. The number of breaths per breath set may be configured to apply to both ascending and descending phases of the multi-breath recruitment maneuver. Alternately, element 914 may be configured to offer an input control for a number of breaths per ascending or descending breath set. Alternately still, element 914 may be configured to offer an input control for a total number of breaths for the multi-breath low flow recruitment maneuver (not shown). Further, the clinician may input a total number of breath sets for the multi-breath recruitment maneuver at element 916. Alternately, the total number of breath sets may be input separately for ascending breath sets and descending breath sets (not shown).

A flow input window 918 may also be provided wherein a clinician may enter a parameter for a peak targeted flow for the multi-breath, low flow recruitment maneuver (not shown). Peak targeted flow is defined as discussed above regarding flow input window 316. In order to deliver a low flow multi-breath recruitment maneuver, the flow may be monitored for each inspiratory and/or expiratory phase (depending on whether the settings require one or both of the phases to be at low flow) of each breath.

In an optional settings input window 924, a window is provided wherein a clinician may enter any number of optional parameters and settings. Optional settings input window 924 is termed optional not to imply in any sense that input into this window is unnecessary or unimportant. Rather, settings entered into this window may not directly relate to the delivery of a multi-breath recruitment maneuver as described herein, but rather may be more applicable to the prescribed ventilation of a patient, and thus, may be entered elsewhere in the configurations settings and screens available to the clinician on the ventilator. However, it may be that one or more parameters are of interest to a particular clinician, or to a standard or hospital protocol as described above. As such, the optional settings input window 924 is provided to allow for input of additional ancillary parameters for monitoring or display during the multi-breath recruitment maneuver. For instance, an alternate value for fractional inspiratory oxygen (FiO₂) may be input into element 926 by the clinician for monitor and control during the recruitment maneuver. Optional settings input window 924 may also provide an option to select element 928, which may store previous multi-breath recruitment maneuver settings, as described above. Additionally, optional settings input window 924 may provide an element to enter a recruitment maneuver hold time (not shown), as described previously with reference to time input window 310. In the case of a multi-breath recruitment maneuver, the hold time may refer not to the target recruitment maneuver pressure, but to the PIP for each inspiratory phase of each breath in the multi-breath maneuver. Other trigger controls, as described above with reference to optional settings input window 322, may also be offered in optional settings input window 924. Further, optional settings input window 924 may provide for other customized settings not previously discussed or disclosed, but desired by a particular clinician and available on other settings screens or displays of the ventilator (not shown). As such, a customized trigger element may be provided for calling on alternate displays and drawing particular parameter settings into the Recruitment Maneuver Input Screen, as desired by a clinician.

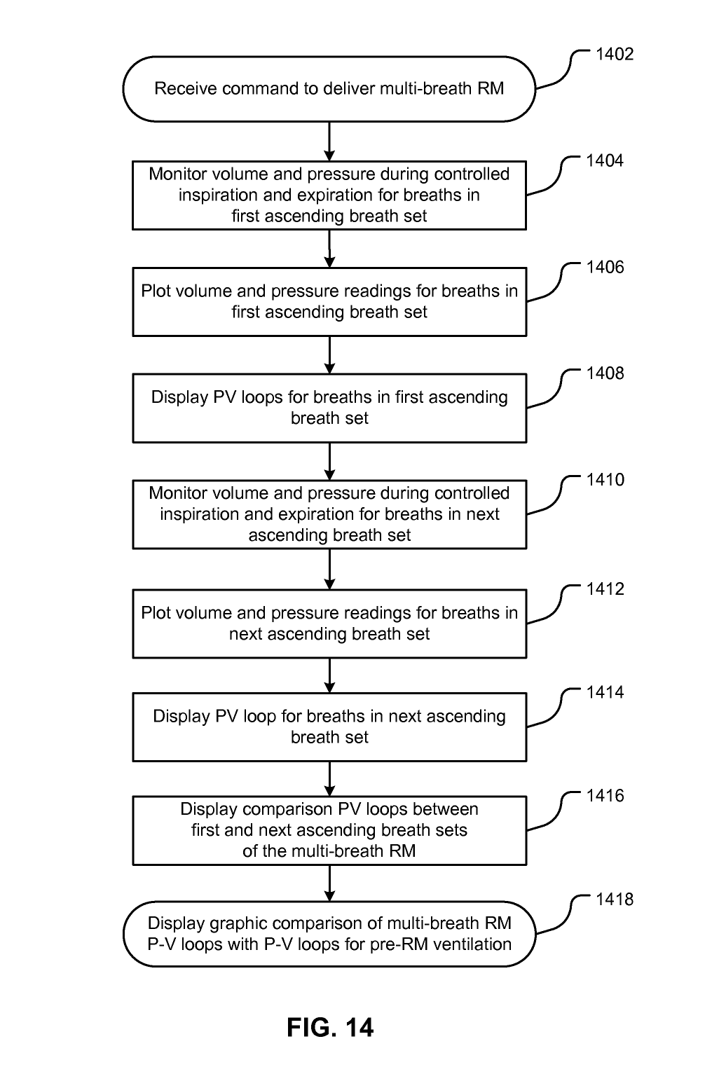

A graphics input window 930 may be provided wherein a clinician may select any number of graphical representations for recruitment maneuver data to be calculated and displayed. Specifically, the clinician may desire a graphical display of a volume curve, a pressure curve (e.g., at element 938), a flow curve, etc., for each breath or each breath set of the recruitment maneuver (not shown). Additionally, the clinician may desire to compare any of these recruitment maneuver curves with curves calculated during prescribed ventilation of a particular patient, either before or after the delivery of the recruitment maneuver (not shown). Further still, a clinician may select an element for a multi-breath recruitment maneuver pressure-volume loop (PV loop) for display. The P/V loop may be generated per breath, at element 932, or it may display data gathered over a breath set, at element 934. The details of the generation and display of one or more PV loops will be discussed further herein with reference to FIG. 14. The clinician may also select a comparison display of a PV loop generated from the multi-breath recruitment maneuver against pre- or post-recruitment maneuver PV loops generated from data during prescribed ventilation of the patient, at element 936. The Multi-Breath RM Input Screen 902 may be further configured to offer other suitable and useful graphical display representations of the recruitment maneuver data, as well as any other suitable comparison display that may be useful to the clinician.

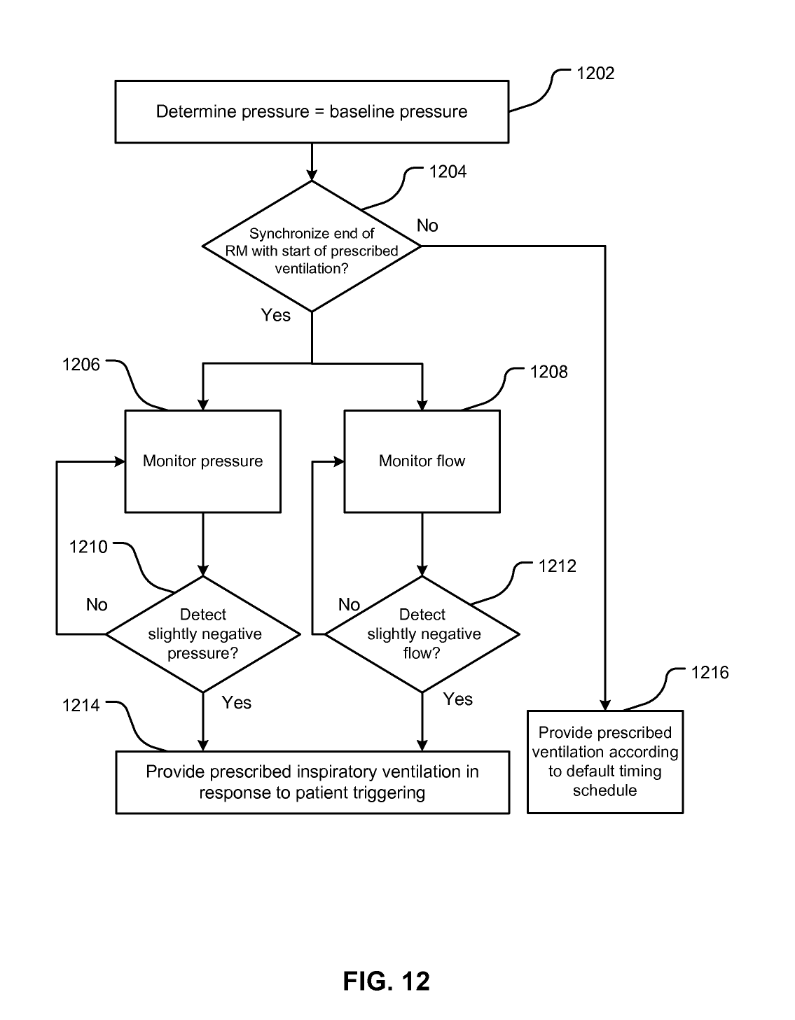

In a prescribed ventilation resume window 940, elements are provided wherein a clinician may configure the post-recruitment maneuver prescribed ventilation to be delivered to a patient. These elements may be defined as described with reference to prescribed ventilation resume window 338 above, for instance, elements 942, 944, and 946, and elements 950 and 952. The clinician may also be able to make a selection at element 948 to synchronize the end of the recruitment maneuver with the beginning of prescribed ventilation inspiration in cases of a spontaneously breathing patient. The applicability for this functionality may be more appropriate for a multi-breath recruitment maneuver where the patient may be more apt to be conscious during the maneuver. Disclosed embodiments of synchronization from the multi-breath recruitment maneuver to the prescribed ventilation of a patient will be discussed in further detail with reference to FIG. 12 below.

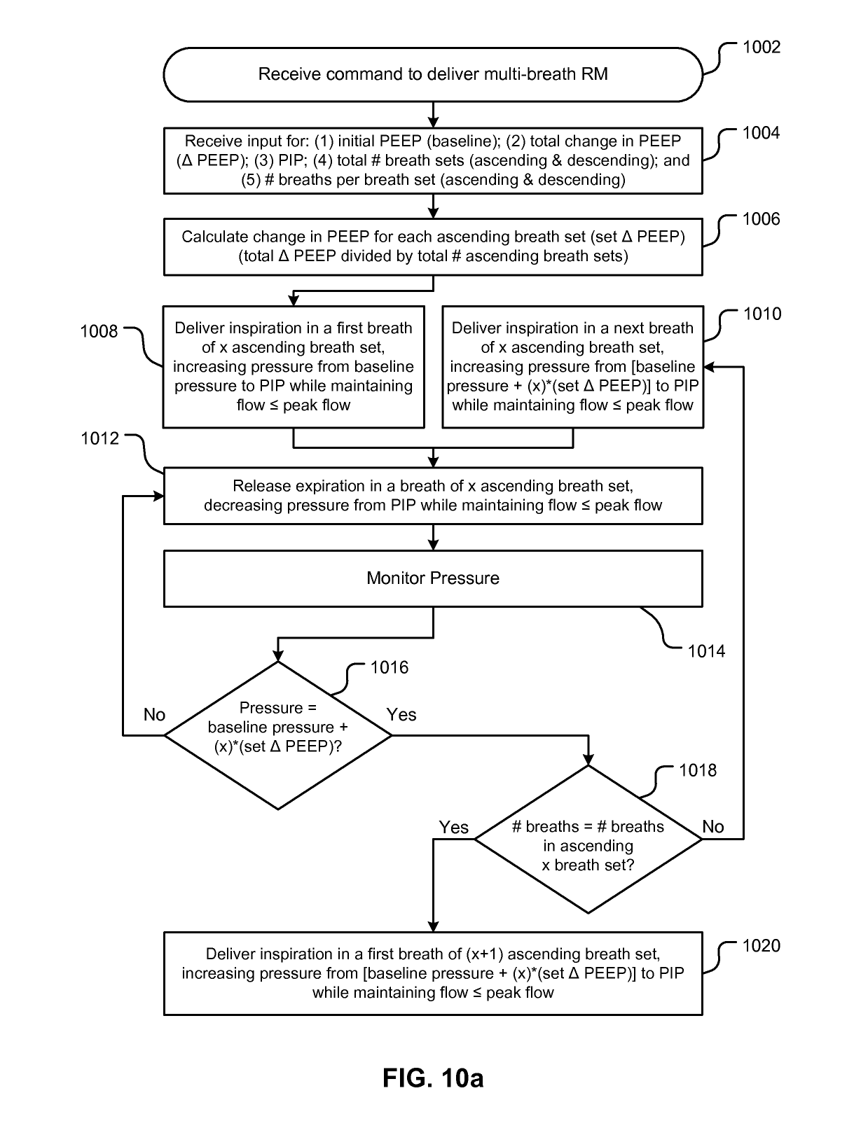

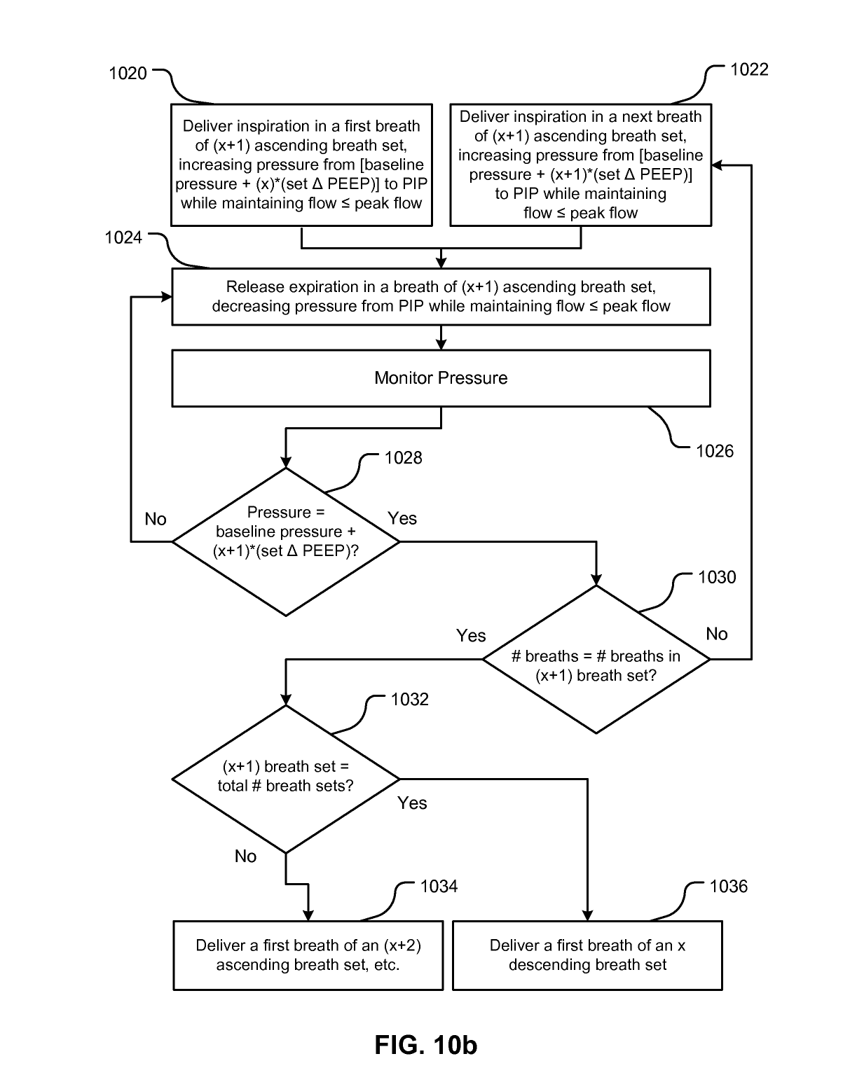

FIGS. 10a and 10b are flow-diagrams illustrating an embodiment of a method for delivering breaths in an ascending phase of a multi-breath, low flow recruitment maneuver, as described herein. These figures disclose only one embodiment of the many potential embodiments described above.

Specifically, FIGS. 10a and 10b illustrate an embodiment of a method for delivering an ascending phase of a multi-breath, low flow recruitment maneuver. The disclosed embodiment maintains a constant PIP throughout the maneuver. In the disclosed embodiment, PEEP is increased an aggregate amount over the ascending phase of the multi-breath recruitment maneuver. Further, the ascending phase is divided into a number of ascending breath sets (xₙ), such that the incremental increase in PEEP for each ascending breath set is equal to the total change in PEEP divided by the total number of ascending breath sets. Further, each ascending breath set includes a certain number of breaths. As illustrated in the disclosed embodiment, each breath within an ascending breath set shares the same incremental change in PEEP. Each breath in each breath set of the ascending phase, according to the disclosed embodiment, is delivered at low flow. Settings for the disclosed embodiment do not include a hold time for the PIP.

At command operation 1002, a command may be received to deliver a multi-breath, low flow recruitment maneuver, as described herein. At input operation 1004, input may be received at any suitable time before the delivery of the disclosed multi-breath recruitment maneuver. Specifically, as described above, the ventilator may receive input, whether pre-configured or manual, including: an initial PEEP (or baseline pressure); a total change in PEEP over the multi-breath recruitment maneuver (total Δ PEEP); a constant peak inspiratory pressure (PIP) (which may include a PIP that is greater than the PIP of the prescribed ventilation); a total number of ascending and/or descending breath sets; and a total number of breaths per ascending and descending breath set.

At calculate operation 1006, the incremental change in PEEP for each ascending breath set is calculated. By way of illustration, in the disclosed embodiment, the incremental PEEP for each ascending breath set (set Δ PEEP) is equivalent to the total change in PEEP divided by the total number of ascending breath sets. For example, for a total of 10 ascending breath sets, set Δ PEEP would be equal to total Δ PEEP divided by 10. As described above, according to the disclosed embodiment of FIGS. 10a and 10b, each breath in a breath set may share the same “set Δ PEEP” value as the other breaths in the breath set (with the exception of the first breath in each set or the last breath in each set, which will be discussed further herein). According to alternate embodiments of a multi-breath recruitment maneuver, for instance, each breath in an ascending phase may incrementally increase PEEP. In this case, the change in PEEP for each breath (breath Δ PEEP) would be equivalent to the total increase in PEEP divided by the total number of breaths (and, correspondingly for the descending phase, the total decrease in PEEP divided by the total number of breaths).

At first breath x ascending breath set inspiration operation 1008, the ventilator may deliver the first breath of an ascending breath set (for instance, the “x” ascending breath set). The first breath may begin inspiration at the initial baseline pressure if the x ascending breath set is the first breath set, for example, and increase pressure to PIP. A pressure monitor module, or other suitable operation or module, may determined when pressure is equal to the target PIP for each breath inspiration. The FIGS. 10a and 10b utilize the term “baseline” pressure to mean a previous baseline pressure immediately prior to the beginning of the x ascending breath set.

At next breath x ascending breath set inspiration operation 1010, note that each additional breath (next breath) in the x ascending breath set may begin inspiration from the baseline pressure plus the “set Δ PEEP” increment of the x ascending breath set. Alternately, the embodiment described herein may be altered such that the last breath of each set completes expiration at the next breath set baseline pressure (i.e., PEEP+(x+1)*(set Δ PEEP)). In this case, the last breath in each set may complete expiration at a different baseline from the other breaths in that set, and the first breath in each next set would initiate from the baseline pressure indicated for that set (i.e., PEEP+(x+1)*(set Δ PEEP), according to the example above). In either case, according to the disclosed embodiment, for the first breath x ascending breath set inspiration operation 1008 and the next breath x ascending breath set inspiration operation 1010 inspiration is delivered at a flow less than or equal to the peak targeted flow (as determined according to previous embodiments and discussions herein).

At breath x ascending breath set expiration operation 1012, the ventilator may initiate an expiratory phase of a breath (first or next) in the x ascending breath set. Expiration of a breath in the x ascending breath set may decrease from PIP at a flow less than or equal to the peak targeted flow. According to the present embodiment, PIP is a constant, but note that according to other embodiments, PIP may also be increased by a particular increment at each breath set, or otherwise. In that case, expiration would begin from the PIP plus any appropriate multiple x of “set Δ PIP” attributable to the x ascending breath set. As will be apparent to those skilled in the art, a great number of variations are possible, and this example is mentioned only to highlight how alternate embodiments may specifically depart from the present figure and discussion.

At pressure monitor operation 1014, pressure may be monitored by any appropriate means, by any appropriate module or component, as described above.

At pressure determination operation 1016, the ventilator may determine whether the expiratory pressure of a breath in the x ascending breath set is about equal to the baseline pressure plus “x” times the “set Δ PEEP.” The ventilator may determine the pressure by any appropriate method or calculation, as described above. Specifically, if x is the third breath set, the adjusted PEEP would equal initial baseline pressure plus 3*(set Δ PEEP). Additionally, using the example of 10 total breath sets from above, the baseline pressure for x breaths would be [3/10*(total Δ PEEP)+initial PEEP]. If it is determined that pressure is not about equal to baseline pressure+x*(set Δ PEEP), the process may return to the breath x ascending breath set expiration operation 1012, wherein additional expiratory pressure is released at low flow. If it is determined at pressure determination operation 1016 that the pressure is about equal to baseline pressure+x*(set Δ PEEP), the process may proceed to breath determination operation 1018.

At breath determination operation 1018, the ventilator may determine whether the number of breaths delivered in the x ascending breath set is equal to the total number of breaths included the x ascending breath set. As described above, the breath monitor module 830, or some other suitable module or component, may be responsible for determining the number of breaths that have been delivered, and further, whether the total number of breaths in a given breath set have been delivered. If the number of breaths delivered in the x ascending breath set is not equal to the total number of breaths included the x ascending breath set, the process may return to next breath x inspiration operation 1010, wherein a next breath in the x ascending breath set is delivered. If the number of breaths delivered in the x ascending breath set is equal to the total number of breaths included the x ascending breath set, the process may proceed to first breath (x+1) ascending breath set inspiration operation 1020.

At first breath (x+1) ascending breath set inspiration operation 1020, inspiration for a first breath in an (x+1) ascending breath set is delivered from a [baseline pressure+x*(set Δ PEEP)] to PIP. A pressure monitor module, or other suitable operation or module, may determined when pressure is equal to the target PIP for each breath inspiration. Note, again, that as this breath represents the first breath in a breath set, it begins inspiration from the baseline pressure calculation of the previous ascending breath set. Note also that according to the present embodiment, inspiration for each breath is delivered at low flow, i.e., flow is less than or equal to a peak targeted flow, as described above.

FIG. 10b begins at the first breath (x+1) ascending breath set inspiration operation 1020, as described above, so as to clarify that FIG. 10b is merely a continuation of the present embodiment from FIG. 10a.

At next breath (x+1) ascending breath set inspiration operation 1022, note that any additional breaths in the (x+1) ascending breath set initiate inspiration from [baseline pressure+(x+1)*(set Δ PEEP)], as discussed above.

At breath (x+1) ascending breath set expiration operation 1024, as described above for the breath x ascending breath set expiration operation 1012, the ventilator may initiate an expiratory phase of a breath in the (x+1) ascending breath set. Expiration of a breath in the (x+1) ascending breath set may decrease from PIP at a flow less than or equal to the peak targeted flow.

At pressure monitor operation 1026, pressure is monitored, as described above at pressure monitor operation 1014.

At pressure determination operation 1028, the ventilator determines whether the expiratory pressure of a breath of the (x+1) ascending breath set is about equal to a [baseline pressure+(x+1)*(set Δ PEEP)], as described above with reference to pressure determination operation 1016. If the expiratory pressure of a breath of the (x+1) ascending breath set is not about equal to a [baseline pressure+(x+1)*(set Δ PEEP)], the process may return to breath (x+1) ascending breath set expiration operation 1024, where additional expiratory pressure is released at low flow. If the expiratory pressure of the breath of the (x+1) ascending breath set is about equal to a [baseline pressure+(x+1)*(set Δ PEEP)], the process may proceed to breath determination operation 1030.

At breath determination operation 1030, the ventilator may determine whether the number of breaths delivered in the (x+1) ascending breath set is equal to the total number of breaths included the (x+1) ascending breath set, as described with reference to breath determination operation 1018. If the number of breaths delivered in the (x+1) ascending breath set is not equal to the total number of breaths included the (x+1) ascending breath set, the process may return to next breath (x+1) ascending breath set inspiration operation 1022, wherein a next breath in the (x+1) ascending breath set is delivered. Alternately, if the number of breaths delivered in the (x+1) ascending breath set is equal to the total number of breaths included the (x+1) ascending breath set, the process may continue to ascending breath set determination operation 1032.

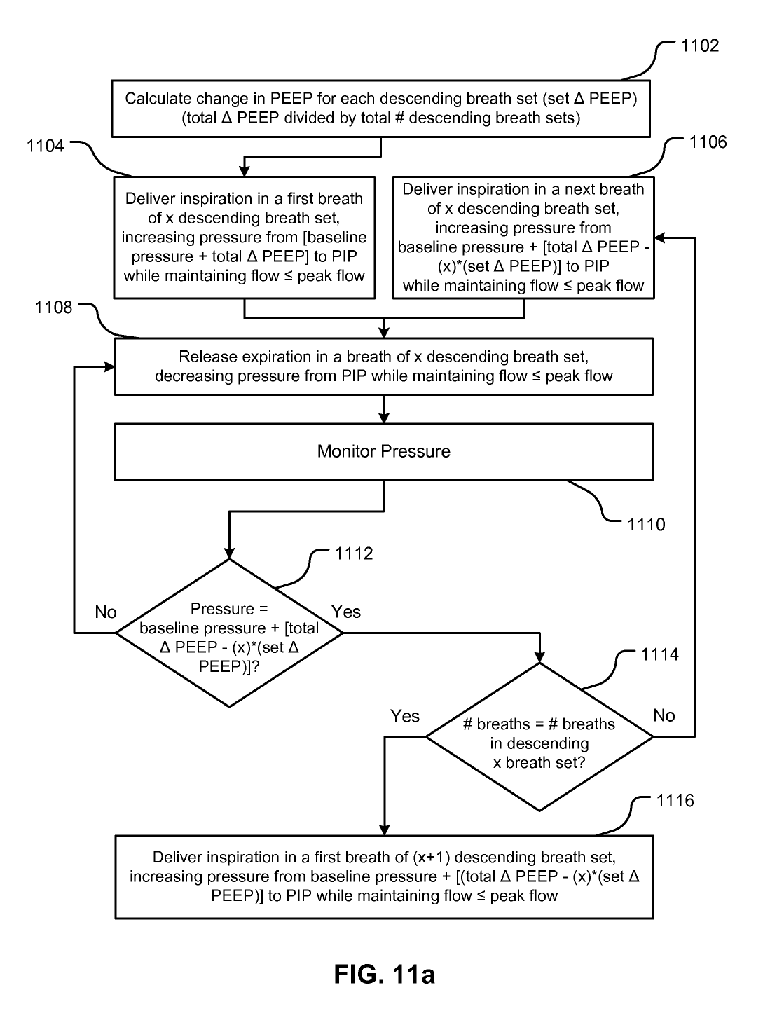

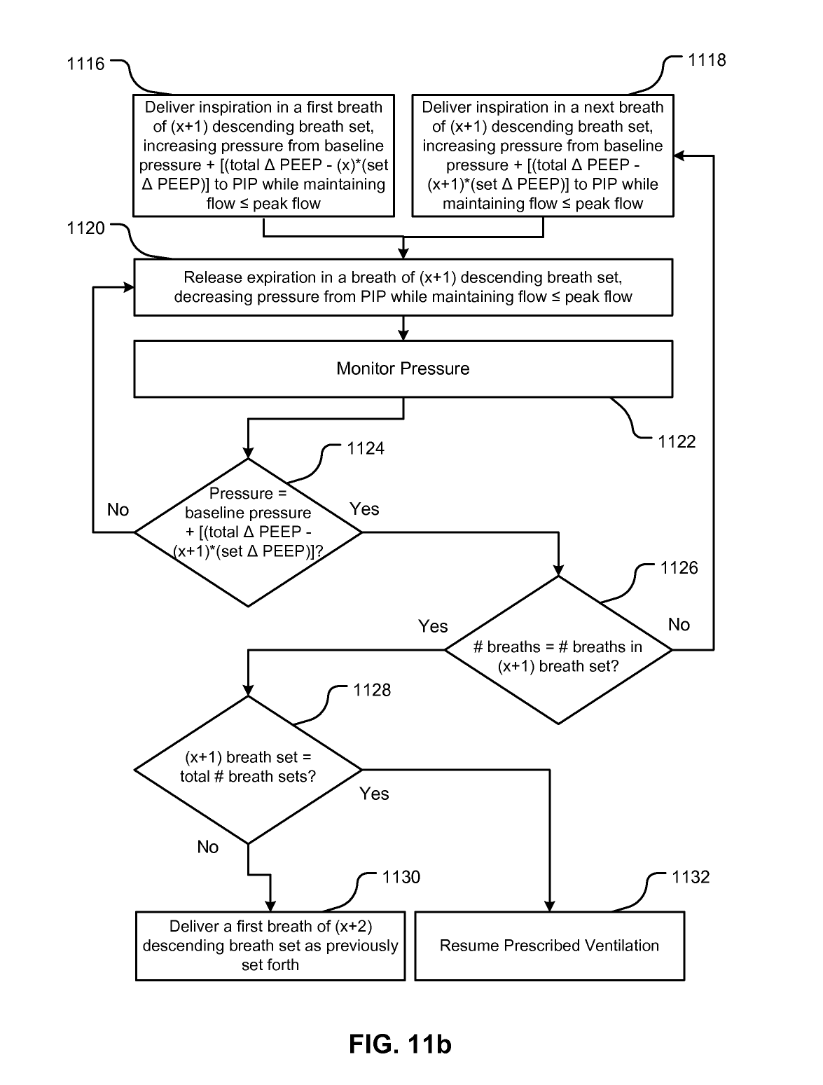

At the ascending breath set determination operation 1032, the ventilator may determine whether the (x+1) ascending breath set is the final breath set of the total number of ascending breath sets. As described above with reference to the breath monitor module 830, this module or any other suitable module, may determine when each ascending breath set is completed and whether the total number of ascending breath sets has been completed. Note that this step may be equally appropriate after breath determination operation 1018 of FIG. 10a and was neglected merely for simplicity of the present embodiment. If the (x+1) ascending breath set is not the final breath set of the total number of ascending breath sets, the process may continue to first breath (x+2) ascending breath set inspiration operation 1034 to deliver a first breath of the (x+2) ascending breath set, and so forth as described for the x and (x+1) ascending breath sets above. Alternately, if the (x+1) ascending breath set is the final breath set of the total number of ascending breath sets, the process may continue to first breath x descending breath set inspiration operation 1104 and deliver a first breath of an x descending breath set for the multi-breath recruitment maneuver, as described below with reference to FIGS. 11a and 11b.

FIGS. 11a and 11b are flow-diagrams illustrating an embodiment of a method for delivering breaths in a descending phase of a multi-breath, low flow recruitment maneuver as described herein.

Specifically, FIGS. 11a and 11b illustrate an embodiment of a method for delivering a descending phase of a multi-breath, low flow recruitment maneuver. In the disclosed embodiment, PEEP is decreased by the same amount as it was increased in the ascending phase of the multi-breath recruitment maneuver. Further, the descending phase is divided into a number of descending breath sets (xₙ), such that the incremental decrease in PEEP for each descending breath set is equal to the total decrease in PEEP (back to the initial PEEP, or initial baseline) divided by the total number of descending breath sets. Further, each descending breath set includes a certain number of breaths. As illustrated in the disclosed embodiment, each breath in a descending breath set shares the same incremental decrease in PEEP. Each breath in each breath set of the descending phase, according to the disclosed embodiment, is delivered at low flow.

At calculate operation 1102 of FIG. 11a, the incremental change in PEEP for each breath set is calculated. The incremental PEEP for each descending breath set (set Δ PEEP) is equivalent to the total change in PEEP divided by the total number of descending breath sets, as described above with reference to calculate operation 1006. The number of breaths and descending breath sets do not need to be the same as the number of breaths and ascending breath sets. However, note that the total Δ PEEP may generally remain the same (the total increment up in PEEP may generally be equivalent to the change back down to an initial baseline pressure). Thus, from the previous example, for a total Δ PEEP, and 11 descending breath sets, the set Δ PEEP for the descending phase would be total Δ PEEP divided by 11 (in which case, the magnitude of PEEP increments downward for each descending breath set would be slightly less than that of the ascending breath set increments upward). Note further, that it is well within the spirit of the present disclosure to enable the clinician to choose an alternate post-recruitment maneuver PEEP (in which case, the total descending Δ PEEP would be equivalent to the ascending Δ PEEP plus or minus an adjustment for the altered post-recruitment maneuver PEEP).

At first breath x descending breath set inspiration operation 1104, the ventilator may deliver inspiration for the first breath of a descending breath set (for instance, the “x” descending breath set). The first breath may begin inspiration at the initial baseline pressure+total Δ PEEP of the ascending phase and increase pressure to PIP. A pressure monitor module, or other suitable operation or module, may determined when pressure is equal to the target PIP for each breath inspiration. Again, at next breath x descending breath set inspiration operation 1106, note that each additional breath (next breath) in the x descending breath set may begin inspiration from the baseline pressure plus the total Δ PEEP minus “x” times a “set Δ PEEP” decrease for the x descending breath set (i.e., baseline pressure+[total Δ PEEP−(x)*(set Δ PEEP)]). Note further that, as discussed above, the step down in PEEP may also be configured to occur at the last breath of a descending breath set. For both the first breath x descending breath set inspiration operation 1104 and the next breath x descending breath set inspiration operation 1106, note that according to the present embodiment inspiration is delivered at a flow less than or equal to the peak targeted flow (as determined according to previous embodiments and discussions herein).

At breath x descending breath set expiration operation 1108, the ventilator may initiate an expiratory phase of a breath in the x descending breath set from PIP, while maintaining flow less than or equal to the peak targeted flow, as described above with reference to breath x ascending breath set expiration operation 1012.

At pressure monitor operation 1110, pressure may be monitored by any appropriate means, by any appropriate module or component, as described above.

At pressure determination operation 1112, the ventilator may determine whether the expiratory pressure of a breath in the x descending breath set is about equal to the baseline pressure+[total Δ PEEP−(x)*(set Δ PEEP)]. The ventilator may determine the pressure by any appropriate method or calculation, as described above at pressure determination operation 1016. If it is determined that pressure is not about equal to baseline pressure+[total Δ PEEP−(x)*(set Δ PEEP)], the process may return to breath x descending breath set expiration operation 1108, wherein additional expiratory pressure is released at low flow. If it is determined that the pressure is about equal to baseline pressure+[total Δ PEEP−(x)*(set Δ PEEP)], the process may proceed to breath determination operation 1114.

At breath determination operation 1114, the ventilator may determine whether the number of breaths delivered in the x descending breath set is equal to the total number of breaths included the x descending breath set, as described above with reference to breath determination operation 1018. If the number of breaths delivered in the x descending breath set is not equal to the total number of breaths included the x descending breath set, the process may proceed to next breath x descending breath set inspiration operation 1106, wherein a next breath in the x descending breath set is delivered. If the number of breaths delivered in the x descending breath set is equal to the total number of breaths included in the x descending breath set, the process may proceed to first breath (x+1) descending breath set inspiration operation 1116.

At first breath (x+1) descending breath set inspiration operation 1116, inspiration for a first breath in an (x+1) descending breath set is delivered from a baseline pressure+[total Δ PEEP−(x)*(set Δ PEEP)] to PIP (wherein a determination that PIP is reached may be accomplished by any suitable pressure monitor module or operation). Note, again, that as this breath represents the first breath in a breath set, it begins inspiration from the baseline pressure calculation of the previous descending breath set. Note also that according to the present embodiment, inspiration for each breath is delivered at low flow, i.e., flow is less than or equal to a peak targeted flow, as described above.

FIG. 11b begins at first breath (x+1) descending breath set inspiration operation 1116, as described above, so as to clarify that FIG. 11b is merely a continuation of the present embodiment from FIG. 11a.

At next breath (x+1) descending breath set inspiration operation 1118, note that any additional breaths in the (x+1) descending breath set initiate inspiration from baseline pressure+[total Δ PEEP−(x+1)*(set Δ PEEP)], as discussed above.

At breath (x+1) descending breath set expiration operation 1120, as described above at breath x ascending breath set expiration operation 1108, the ventilator may initiate an expiratory phase of a breath in the (x+1) descending breath set. Expiration of the breath in the (x+1) descending breath set may decrease from PIP at a flow less than or equal to the peak targeted flow.

At pressure monitor operation 1122, pressure is monitored, as described above at pressure monitor operation 1110.

At pressure determination operation 1124, the ventilator determines whether the expiratory pressure of the breath of the (x+1) descending breath set is about equal to a baseline pressure+[total Δ PEEP−(x+1)*(set Δ PEEP)]. If the expiratory pressure of the breath of the (x+1) descending breath set is not about equal to a baseline pressure+[total Δ PEEP−(x+1)*(set Δ PEEP)], the process may return to breath (x+1) descending breath set expiration operation 1120, where additional expiratory pressure is released at low flow. If the expiratory pressure of the breath of the (x+1) descending breath set is about equal to a baseline pressure+[total Δ PEEP−(x+1)*(set Δ PEEP)], the process may proceed to breath determination operation 1126.

At breath determination operation 1126, the ventilator may determine whether the number of breaths delivered in the (x+1) descending breath set is equal to the total number of breaths included the (x+1) descending breath set, as described above. If the number of breaths delivered in the (x+1) descending breath set is not equal to the total number of breaths included the (x+1) descending breath set, the process may return to next breath (x+1) descending breath set inspiration operation 1118, wherein a next breath in the (x+1) descending breath set is delivered. Alternately, if the number of breaths delivered in the (x+1) descending breath set is equal to the total number of breaths included the (x+1) descending breath set, the process may continue to descending breath set determination operation 1128.

At descending breath set determination operation 1128, the ventilator may determine whether the (x+1) descending breath set is the final breath set of the total number of descending breath sets. If the (x+1) descending breath set is not the final breath set of the total number of descending breath sets, the process may continue to first breath (x+2) descending breath set inspiration operation 1130 to deliver a first breath of the (x+2) descending breath set, and so forth as described for the x and (x+1) descending breath sets above. Alternately, if the (x+1) descending breath set is the final breath set of the total number of descending breath sets, the process may continue to resume prescribed ventilation operation 1132 wherein the multi-breath recruitment maneuver is completed and prescribed ventilation may be resumed, as described above.

FIG. 12 is a flow-diagram illustrating an embodiment of a method for synchronizing the end of a recruitment maneuver with the initiation of a first inspiratory breath of post-recruitment maneuver prescribed ventilation.

At pressure determination operation 1202, the end of a recruitment maneuver is achieved and the pressure is returned to the baseline pressure.

At synchronization determination operation 1204, the ventilator determines whether the recruitment maneuver has been configured to synchronize the end of the recruitment maneuver with the initiation of prescribed ventilation. As described above with reference to the recruitment maneuver input screens, the ventilator may receive a command to synchronize the recruitment maneuver by any suitable mechanism. If the recruitment maneuver should not be synchronized, the process may continue to default timing prescribed ventilation operation 1216 where prescribed ventilation may be resumed according to any suitable settings, pre-configured or manual, according to any appropriate default timing schedule. If the recruitment maneuver should be synchronized, the process proceeds to either synchronization pressure monitor operation 1206 or synchronization flow monitor operation 1208, depending on the desires of the clinician and/or the capabilities of the ventilator.

For a spontaneously breathing patient, the ventilator may be configured to initiate an inspiratory breath in response to a patient trigger. Different embodiments may include either a pressure monitoring method or a flow monitoring method. At synchronization pressure monitor operation 1206, a pressure-triggering method may involve the ventilator monitoring the circuit pressure, as described above, and detecting a slight negative circuit pressure at negative pressure determination operation 1210. The slightly negative circuit pressure may indicate that the patient's respiratory muscles, Pₘ from above, are creating a slight negative pressure gradient between the patient's lungs and the airway opening in an effort to inspire. The ventilator may then initiate an inspiratory breath of the prescribed ventilation at a synchronized prescribed ventilation operation 1214 in response to the pressure-trigger initiated by the patient.

Alternately, at synchronization flow monitor operation 1208, the ventilator may detect a flow-triggered event. Specifically, the ventilator may monitor the circuit flow, as described above. If the ventilator detects a slightly negative flow at negative flow determination operation 1212, this may indicate, again, that the patient is attempting to inspire. In this case, the ventilator is detecting a slightly negative flow into the patient's lungs (in response to a slightly negative pressure gradient as discussed with reference to pressure gradients above). As with the pressure-triggered method, the ventilator may then initiate an inspiratory breath of the prescribed ventilation at synchronized prescribed ventilation operation 1214 in response to the flow-trigger initiated by the patient.

The embodiments described with reference to the above synchronization are not exhaustive and any other suitable method may be utilized to synchronize the end of a recruitment maneuver with prescribed ventilation. Additionally, individual breaths of the multi-breath recruitment maneuver may be synchronized with triggers initiated by a spontaneously breathing patient. As a result of the low flow methods for recruitment maneuver delivery described herein, it may be that low flow recruitment maneuvers are better adapted for delivery to conscious and spontaneously breathing patients. It is in an effort to promote the comfort of a spontaneously breathing patient that the present synchronization methods are proposed.

It will be clear that the systems and methods described herein for a multi-breath low flow recruitment maneuver are well adapted to attain the ends and advantages mentioned as well as those inherent therein. Those skilled in the art will recognize that the methods and systems within this specification may be implemented in many manners and as such is not to be limited by the foregoing exemplified embodiments and examples. In other words, functional elements being performed by a single or multiple components, in various combinations of hardware and software, and individual functions can be distributed among software applications at either the client or server level. In this regard, any number of the features of the different embodiments described herein may be combined into one single embodiment and alternate embodiments having fewer than or more than all of the features herein described are possible.

While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the appended claims.

### Pressure-Volume Loop Generation

Pressure-volume loops, as have been described briefly above, may provide useful clinical and diagnostic information to physicians regarding patients on mechanical ventilators. More specifically, pressure-volume loops may be an indication and graphical representation of the respiratory compliance of a patient.

Respiratory compliance refers to the ease with which the respiratory system expands or distends. Respiratory compliance is the inverse of elastance, which refers to the respiratory system's tendency to return to its baseline form. Respiratory resistance encompasses the frictional forces attributable to the anatomical (e.g., oral, tracheal, bronchial, etc.) and mechanical (e.g., tubing, etc.) structures, and also the viscous forces attributable to the lungs, tissues, and organs, that “resist” or impede airflow to and from the alveoli of the lungs. Respiratory compliance and resistance together represent the “load” against which the ventilator and the patient's muscles must work to deliver air to the patient's lungs. As will be discussed further herein, a pressure-volume loop generated during inspiratory and expiratory phases of ventilation may indicate the respiratory compliance of a given patient.

Specifically, a pressure-volume loop (PV loop) provides a visual representation, in the area between the inspiratory plot of pressure vs. volume and the expiratory plot of pressure vs. volume, that indicates respiratory compliance. Further, PV loops may be compared to one another to determine whether compliance has changed. Relevant to the present disclosure, a PV loop generated during prescribed ventilation prior to a recruitment maneuver may be compared to a PV loop generated during or after a recruitment maneuver.

In a further embodiment, PV loops may be useful for suggesting to a clinician a minimum PEEP and/or a maximum PIP for prescribed ventilation of a particular patient. The suggestion may be based on inflection points of the PV loop (if any). Additionally, or alternately, the suggestion may be based on a determination of optimal compliance for the particular patient. Optimal compliance may be determined from a PV loop itself. Alternately, in the case of a multi-breath recruitment maneuver, optimal compliance may be determined as the dynamic compliance over the multi-breath recruitment maneuver.

In another embodiment, PV loops for a particular patient may be archived over the duration of the particular patient's treatment. A clinician may then select a trend feature for viewing the stored PV loops. Viewing may further involve scrolling through each PV loop manually or initiating a “play” feature wherein the stored PV loops may be visualized in order of their generation over time using the same pressure and volume scale for each loop. Among other things, this feature may enable the clinician to evaluate the progress of the particular patient's condition over the duration of the respiratory treatment.

The following figures illustrate embodiments of displaying and utilizing PV loops in connection with the present methods and systems.

FIG. 13 is a flow-diagram illustrating an embodiment of a method for generating a pressure-volume loop during a single-breath, low flow recruitment maneuver as described herein.

At command operation 1302, any suitable command is received to deliver a single-breath, low flow recruitment maneuver, as described herein. At low flow inspiration operation 1304, a low flow inspiratory phase of a single-breath recruitment maneuver is initiated.

At inspiratory monitor operation 1306, the ventilator may monitor pressure and volume during the delivery of the recruitment maneuver. Although the above disclosure has primarily focused on monitoring pressure during the different phases and steps of the disclosed delivery of a recruitment maneuver, monitoring volume is well within the spirit of the present application. Volume may thus be monitored by any suitable method, or may alternately be derived according to the ventilatory equation above. Further, as mentioned with respect to the modules, displays, and input controls described above, monitoring circuit volume may be easily added and configured into the present systems and methods.

At inspiratory store operation 1308, the ventilator may store the pressure data gathered or derived by the ventilator versus the volume data gathered or derived by the ventilator during the inspiratory phase of the recruitment maneuver. As described above regarding the various graphical representations and displays available to the clinician, for example with reference to the recruitment maneuver input screens, plotting pressure data versus volume data is well within the disclosed capabilities of the ventilator.

At inspiratory pressure determination operation 1310, the ventilator may determine that the pressure is equal to a target recruitment maneuver pressure, as disclosed herein. At this point, the volume and pressure should merge at the peak inspiratory pressure point (or, target recruitment maneuver pressure).

At low flow expiration operation 1312, the ventilator may begin to release the expiratory pressure while maintaining the flow less than or equal to the peak targeted flow.

At expiratory monitor operation 1314, pressure and volume continue to be monitored, as described above.

At expiratory store operation 1316, the ventilator may store the pressure data versus the volume data gathered by the ventilator during the expiratory phase of the single-breath recruitment maneuver.

At expiratory pressure determination operation 1318, the ventilator may determine that the pressure is about equal to the baseline pressure and that the recruitment maneuver is complete.

At graphical display operation 1320, the ventilator may graphically display, by any suitable means, the PV loop generated from the data gathered during the single-breath recruitment maneuver.

At comparison graphical display operation 1322, the ventilator may alternately or additionally graphically display, by any suitable means, the PV loop generated from the data gathered during the single-breath recruitment maneuver versus a PV loop generated from pressure and volume data gather during prescribed ventilation, either before or after the recruitment maneuver. As will be understood by those skilled in the art, it may be useful to a clinician to visualize whether the respiratory compliance of the lungs increased after the delivery of a recruitment maneuver.

FIG. 14 is a flow-diagram illustrating an embodiment of a method for generating a pressure-volume loop during a multi-breath, low flow recruitment maneuver as described herein.

At command operation 1402, any suitable command is received to deliver a multi-breath, low flow recruitment maneuver, as described herein.

At first ascending breath set monitor operation 1404, the ventilator may monitor and store volume and pressure during the first ascending breath set of a multi-breath recruitment maneuver, as disclosed above.

At plot first ascending breath set operation 1406, the ventilator may plot the pressure data versus the volume data monitored by the ventilator during the first ascending breath set of a multi-breath recruitment maneuver.

At display PV loops operation 1408, the ventilator may display PV loops generated for breaths of the first ascending breath set. According to embodiments, the ventilator may display PV loops for one or more of the breaths of the first ascending breath set.

At next ascending breath set monitor operation 1410, the ventilator may monitor and store volume and pressure during a next ascending breath set of a multi-breath recruitment maneuver, as disclosed above.

At plot next ascending breath set operation 1412, the ventilator may plot the pressure data versus the volume data monitored by the ventilator during the next ascending breath set of a multi-breath recruitment maneuver.

At display PV loops operation 1414, the ventilator may display PV loops generated for breaths of the next ascending breath set. According to embodiments, the ventilator may display PV loops for one or more of the breaths of the next ascending breath set.

At comparison first vs. next graphical display operation 1416, the ventilator may provide a comparison of the different PV loops generated by plotting the pressure data versus the volume data gathered by the ventilator during the first and the next ascending breath sets of a multi-breath recruitment maneuver.

At comparison pre- vs. RM graphical display operation 1418, the ventilator may provide a comparison of the different PV loops generated during a multi-breath recruitment maneuver with PV loops generated during pre- (or, alternately, post-) recruitment maneuver prescribed ventilation.

As will be apparent to one skilled in the art, comparison PV loops may be similarly generated between the ascending and descending phases of a multi-breath recruitment maneuver, between breaths in the descending phase of a multi-breath recruitment maneuver, and/or between a PV loop generated by averaging the data over ascending and descending phases, breath sets, and/or breaths of the whole multi-breath recruitment maneuver with pre- or post-RM generated PV loops.

It will be clear that the systems and methods described herein are well adapted to attain the ends and advantages mentioned as well as those inherent therein. Those skilled in the art will recognize that the methods and systems within this specification may be implemented in many manners and as such is not to be limited by the foregoing exemplified embodiments and examples. In other words, functional elements being performed by a single or multiple components, in various combinations of hardware and software, and individual functions can be distributed among software applications at either the client or server level. In this regard, any number of the features of the different embodiments described herein may be combined into one single embodiment and alternate embodiments having fewer than or more than all of the features herein described are possible.

While various embodiments have been described for purposes of this disclosure, various changes and modifications may be made which are well within the scope of the present invention. Numerous other changes may be made which will readily suggest themselves to those skilled in the art and which are encompassed in the spirit of the disclosure and as defined in the appended claims.

What is claimed is:

1. A method for generating a pressure-volume loop (PV loop) for a single-breath, low flow recruitment maneuver, the method comprising:
monitoring pressure, volume, and flow while delivering air pressure from a baseline pressure to a target recruitment maneuver pressure at low flow;
storing pressure data versus volume data collected during delivery of air pressure from the baseline pressure to the target recruitment maneuver pressure;
upon determining that pressure is about equal to the target recruitment maneuver pressure, releasing air pressure;
monitoring pressure, volume, and flow while releasing air pressure from the target recruitment maneuver pressure to the baseline pressure at low flow;
upon determining that pressure is about equal to the baseline pressure, storing pressure data versus volume data collected during release of air pressure from the target recruitment maneuver pressure to the baseline pressure; and
displaying the stored pressure data versus volume data for the delivering air pressure and the stored pressure data versus volume data for the releasing air pressure to generate a PV loop for the single-breath, low flow recruitment maneuver.

2. The method of claim 1, wherein delivering air pressure at low flow further comprises:
delivering air pressure at a first inspiratory rate;
determining whether flow is less than a peak targeted flow, wherein when flow is not less than the peak targeted flow, delivering air pressure at a second inspiratory rate less than the first inspiratory rate; and
upon determining that the pressure is about equal to the target recruitment maneuver pressure, discontinuing delivering air pressure.

3. The method of claim 2, wherein the peak targeted flow is about 10 liters per minute (lpm).

4. The method of claim 1, wherein releasing air pressure at low flow further comprises:
releasing air pressure at a first expiratory rate;
determining whether flow is less than a peak targeted flow, wherein when flow is not less than the peak targeted flow, releasing air pressure at a second inspiratory rate less than the first inspiratory rate; and
upon determining that the pressure is about equal to the baseline pressure, discontinuing releasing air pressure.

5. The method of claim 1, wherein the peak targeted flow is about 10 liters per minute (lpm).

6. The method of claim 1, further comprising:
displaying the PV loop for the single-breath, low flow recruitment maneuver; and
displaying a PV loop generated during prescribed ventilation before delivery of the single-breath, low flow recruitment maneuver for comparison by a clinician.

7. The method of claim 1, further comprising:
storing a plurality of PV loops during ventilation of a patient; and
displaying each of the plurality of PV loops in succession.

8. The method of claim 1, further comprising calculating an optimal compliance for a patient based on the PV loop.

9. The method of claim 8, further comprising suggesting a minimum positive end expiratory pressure (PEEP) and a peak inspiratory pressure (PIP) based on the optimal compliance.

10. A method for generating a plurality of pressure-volume loops (PV loops) for a multi-breath, low flow recruitment maneuver, the method comprising:
monitoring pressure, volume, and flow while delivering pressure at low flow from a first baseline pressure to a peak inspiratory pressure during each of a first plurality of breaths of an ascending phase of a multi-breath recruitment maneuver;
monitoring pressure, volume, and flow while releasing pressure at low flow from the peak inspiratory pressure to the first baseline pressure during each of the first plurality of breaths of the ascending phase of the multi-breath recruitment maneuver;
storing pressure data versus volume data collected during delivery and release of pressure for each of the first plurality of breaths of the ascending phase of the multi-breath recruitment maneuver;
monitoring pressure, volume, and flow while delivering pressure at low flow from a second baseline pressure to the peak inspiratory pressure during each of a second plurality of breaths of the ascending phase of the multi-breath recruitment maneuver;
monitoring pressure, volume, and flow while releasing pressure at low flow from the peak inspiratory pressure to the second baseline pressure during each of the second plurality of breaths of the ascending phase of the multi-breath recruitment maneuver;
storing pressure data versus volume data collected during delivery and release of pressure for each of the second plurality of breaths of the ascending phase of the multi-breath recruitment maneuver; and
displaying the stored pressure data versus volume data for the first plurality of breaths and the stored pressure data versus volume data for the second plurality of breaths of the ascending phase of the multi-breath recruitment maneuver.

11. The method of claim 10, further comprising:
determining that the second baseline pressure is about equal to the baseline pressure plus a total change in positive end expiratory pressure (PEEP);
monitoring pressure, volume, and flow while delivering pressure at low flow from the first baseline pressure to the peak inspiratory pressure during each of a first plurality of breaths of a descending phase of the multi-breath recruitment maneuver;
monitoring pressure, volume, and flow while releasing pressure at low flow from the peak inspiratory pressure to the first baseline pressure during each of the first plurality of breaths of the descending phase of the multi-breath recruitment maneuver;
storing pressure data versus volume data collected during delivery and release of pressure for each of the first plurality of breaths of the descending phase of the multi-breath recruitment maneuver; and
displaying the stored pressure data versus volume data for the first plurality of breaths of the descending phase of the multi-breath recruitment maneuver.

12. The method of claim 10, wherein delivering pressure at low flow further comprises:
delivering pressure at a first inspiratory rate;
determining whether flow is less than a peak targeted flow, wherein when flow is not less than the peak targeted flow, delivering pressure at a second inspiratory rate less than the first inspiratory rate; and
upon determining that the pressure is about equal to the peak inspiratory pressure, discontinuing the delivering pressure.

13. The method of claim 12, wherein the peak targeted flow is about 10 liters per minute (lpm).

14. The method of claim 10, wherein releasing pressure at low flow further comprises:
releasing pressure at a first expiratory rate;
determining whether flow is less than a peak targeted flow, wherein when flow is not less than the peak targeted flow, releasing pressure at a second inspiratory rate less than the first inspiratory rate; and
upon determining that the pressure is about equal to the first baseline pressure, discontinuing the releasing pressure.

15. The method of claim 14, wherein the peak targeted flow is about 10 liters per minute (lpm).

16. The method of claim 10, further comprising:
displaying the plurality of PV loops for comparison by a clinician, wherein the clinician may visualize at least two or more of the plurality of PV loops concurrently.

17. The method of claim 10, further comprising:
storing the plurality of PV loops during ventilation of a patient; and
displaying each of the plurality of PV loops in succession.

18. The method of claim 10, further comprising calculating a dynamic compliance for a patient based on the plurality of PV loops.

19. The method of claim 18, further comprising suggesting a minimum positive end expiratory pressure (PEEP) and a peak inspiratory pressure (PIP) based on the dynamic compliance.

20. A ventilatory system for displaying a pressure-volume loop (PV loop) for a single-breath, low flow recruitment maneuver, comprising:
at least one display device;
at least one processor; and
at least one memory, communicatively coupled to the at least one processor and containing instructions that, when executed by the at least one processor, perform a method comprising:
monitoring pressure, volume, and flow while delivering pressure from a baseline pressure to a target recruitment maneuver pressure at low flow;
plotting pressure data versus volume data collected during delivery of pressure from the baseline pressure to the target recruitment maneuver pressure;
upon determining that pressure is about equal to the target recruitment maneuver pressure, releasing pressure at low flow;
monitoring pressure, volume, and flow while releasing pressure from the target recruitment maneuver pressure to the baseline pressure at low flow;
upon determining that pressure is about equal to the baseline pressure, plotting pressure data versus volume data collected during release of pressure from the target recruitment maneuver pressure to the baseline pressure; and
displaying on the display device the plotted pressure data versus volume data for the delivering pressure and the plotted pressure data versus volume data for the releasing pressure to generate a PV loop for the single-breath, low flow recruitment maneuver.
